(19) United States
(12) Patent Application Publication
(10) Pub. No.: US 2010/0222332 A1
(43) Pub. Date: Sep. 2, 2010

(54) PYRAZOLO-PYRAZINES DERIVATIVES USED AS G PROTEIN INHIBITORS

(75) Inventors: Olivier Lavergne, Palaiseau (FR); Laetitia Brehu, Les Ulis (FR); Gregoire Prevost, Antony (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Ipsen Pharma S.A.S., Boulogne-Billancourt (FR)

(21) Appl. No.: 12/680,703

(22) PCT Filed: Sep. 25, 2008
(86) PCT No.: PCT/FR2008/001332

§ 371 Date: Mar. 29, 2010

(30) Foreign Application Priority Data
Sep. 27, 2007 (FR) 07/06768

(51) Int. Cl. A61K 31/551; C07D 487/04; A61K 31/4985; A61P 35/00; A61P 33/00; A61P 25/00; A61P 31/12; A61P 37/00; A61P 29/00

(52) U.S. Cl. 514/221; 544/350; 514/249; 540/568

(57) ABSTRACT

The invention relates to pyrazolo-pyrazines derivatives of the general formula (I) in which the radicals Z, R₁, R₂, R₃ and R₄ represent various variable groups, X is a sulphur atom or a selenium atom, and n is an integer equal to 1 or 2. These compounds are inhibitors of G proteins. They are of particular interest for treating diseases in which the heterotrimeric G protein is involved. The invention also relates to pharmaceutical compositions containing said products, and to the use thereof for preparing a drug.

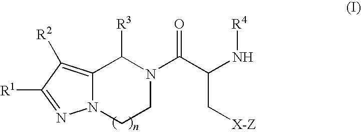

A subject of the present invention is pyrazolo-pyrazine derivatives. These compounds are G protein inhibitors. They are therefore particularly useful for treating the pathologies which result from the involvement of the heterotrimeric G protein. The invention also relates to pharmaceutical compositions containing said products and their use for the preparation of a medicament.

The G proteins are, in fact, the structural association of three distinct sub-units called α, β and γ, but function as dissociable entities constituted by α sub-units on the one hand and β/γ dimers on the other.

The G proteins participate in the transmission of signals outside the cell, thanks to their interaction with receptors with seven transmembrane domains, inside using different effectors including adenylate cyclase, phospholipase C or also the ionic channels. The adenylate cyclase enzyme generates cyclic adenosine monophosphate (cAMP) (cf. Gilman, Biosci. Rep., 15, 65-97 (1995)). Thus it is known, that in order to activate adenylate cyclase, it is necessary for the G proteins to be transitionally in a heterotrimeric form, in which form the monomer constituted by an α sub-unit is associated with the dimer constituted by the β and γ sub-units. It is only in this situation that the signal outside the cell can activate the α sub-unit of a G protein, which can, after disassociation, modulate the adenylate cyclase and modulate the production of cAMP.

It is also known that the β/γ dimers can directly activate effectors leading to the activation of kinases regulated by extracellular signals (ERKs) or MAP kinases. A direct link between the β/γ sub-units and the src or src-like kinases has been demonstrated (cf. Gutkind, J. S. J. Biol. Chem. 273, 1839-1842 (1998)).

Moreover, bacterial toxins such as Vibrio cholera and Bortella pertussis, peptides such as mastoparan and suramin have been presented as directly modulating the activity of the G proteins (cf. Freissmuth, M., Boehm, S., Beindl, W., et al. Mol. Pharmacol. 49, 602-611 (1996); Boehm, S., Huck, S., Motejlek, A., et al. Journal of Neurochemistry 66, 1019-1026 (1996); Cachero, T. G., Rigual, R., Rocher, A. & Gonzalez, C. Eur. J. Neurosci. 8, 2320-2327 (1996); Danilenko, M., Worland, P., Carlson, B., Sausville, E. A. & Sharoni, Y. Biochem. Biophys. Res. Commun. 196, 1296-1302 (1993); Beindl, W., Mitterauer, T., Hohenegger, M., Ijzerman, A. P., Nanoff, C. & Freissmuth, M. Mol. Pharmacol. 50, 415-423 (1996)).

For example, the cholera toxin modifies the α_{S} sub-unit of the G protein by adding an ADP-ribose originating from NAD to an arginine-specific acceptor site. This completely blocks the activity of the GTPase, causing persistent stimulation of its effector following the adenylate cyclase and leading to an overproduction of cAMP.

The harmful effects of an abnormal cAMP level are also known and occur in particular at the level of the following biological functions or disorders: smell, taste, light perception, neurotransmission, neurodegeneration, endocrine and exocrine gland functions, autocrine and paracrine regulation, arterial tension, embryogenesis, benign cell proliferation, oncogenesis, viral infection and immunological functions, diabetes, obesity and pain.

The applicant has just discovered that certain pyrazolo-pyrazine derivatives, namely the compounds of general formula (I) as defined below, can be used to treat or prevent pathologies which result from the involvement of the heterotrimeric G protein.

A subject of the invention is therefore compounds of general formula

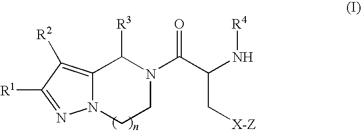

in racemic, enantiomeric or diastereoisomeric form or any combinations of these forms and in which
Z represents a hydrogen atom or a radical of general formula

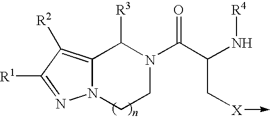

R¹ and R² represent, independently, a hydrogen atom, an aryl or heteroaryl radical, the aryl or heteroaryl radicals being optionally substituted by one or more identical or different substituents chosen from: halo, hydroxy, alkyl, haloalkyl, alkoxy, haloalkoxy, aryl, aryloxy, —NRR′, —C(O)—NRR′, —NR_{N}—C(O)R′, —SO₂—R, —SiRR′R″ or a heterocycloalkyl; or a radical of formula

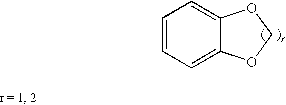

or R¹ and R² form together with the carbon atoms to which they are attached, a cycloalkyl or heterocycloalkyl radical;
R³ represents a (C₁-C₈)alkyl radical or a cycloalkylalkyl, aryl or arylalkyl radical, the aryl group of the aryl and arylalkyl radicals being optionally substituted by one or more identical or different substituents chosen from: halo, hydroxy, alkyl, haloalkyl, alkoxy, haloalkoxy, aryl, aryloxy, —NRR′, —C(O)—NRR′, —NR_{N}—C(O)R′, —SO₂—R, —SiRR′R″ or a heterocycloalkyl;
R_{N} represents a hydrogen atom or an alkyl radical;
R, R′ and R″ represent, independently, an alkyl or aryl radical;
R⁴ represents a hydrogen atom or a radical of formula —CO—O—R⁵;
R⁵ represents an alkyl or arylalkyl radical;
n represents the integer 1 or 2;
X represents a sulphur atom or a selenium atom; or a pharmaceutically acceptable salt of the latter.

In the definitions given above, the expression halo (halogeno) represents the fluoro, chloro, bromo or iodo, preferably fluoro, chloro or bromo radical.

By alkyl, unless otherwise specified, is meant a linear or branched alkyl radical with 1 to 6 carbon atoms such as for example the methyl, ethyl, propyl, isopropyl, butyl, isobutyl, sec-butyl and tert-butyl, pentyl, neopentyl, isopentyl, hexyl, isohexyl radicals. The term (C₁-C₈)alkyl designates an alkyl radical having 1 to 8 carbon atoms, linear or branched, such as the radicals containing 1 to 6 carbon atoms as defined above but also the linear or branched radicals containing 7 or 8 carbon atoms such as for example heptyl, octyl, 1,1,2,2-tetramethyl-propyl, 1,1,3,3-tetramethyl-butyl. By C₄-C₈ alkyl, is meant an alkyl radical as defined above and containing 4 to 8 carbon atoms. By haloalkyl, is meant an alkyl radical in which at least one of the hydrogen atoms (and optionally all) is replaced by a halo radical such as for example trifluoromethyl, dichloroethyl.

The term alkoxy designates the radicals in which the alkyl radical is as defined above such as for example the methoxy, ethoxy, propyloxy or isopropyloxy radicals but also secondary or tertiary linear butoxy, pentyloxy. By haloalkoxy, is meant an alkoxy radical in which at least one of the hydrogen atoms (and optionally all) is replaced by a halo radical such as for example trifluoromethoxy, dichloroethoxy.

The term cycloalkyl (or ring) designates a saturated carbon monocyclic system comprising 3 to 7 carbon atoms, and preferably the cyclopropyl, cyclobutyl, cyclopentyl, cyclohexyl or cycloheptyl rings. The term cycloalkylalkyl preferably designates the radicals in which the cycloalkyl and alkyl radicals are as defined above such as for example cyclohexyl-methyl, cyclohexyl-ethyl, cyclopropyl-methyl.

The expression heterocycloalkyl (or heterocycle) designates a condensed monocyclic or bicyclic saturated system comprising 2 to 6 carbon atoms and at least one heteroatom. This radical can contain several identical or different heteroatoms. Preferably, the heteroatoms are chosen from oxygen, sulphur or nitrogen. As examples of heterocycloalkyls, there may be mentioned pyrrolidine, imidazolidine, pyrrazolidine, isothiazolidine, thiazolidine, isoxazolidine, oxazolidine, piperidine, piperazine, azepane (azacycloheptane), azacyclooctane, diazepane, morpholine, decahydroisoquinoline (or decahydroquinoline), tetrahydrofuran or tetrahydrothiophene.

The expression aryl represents an aromatic radical, constituted by a ring or by 2 to 3 condensed rings, such as for example the phenyl, naphthyl or fluorenyl radical. The term aralkyl (arylalkyl) preferably designates the radicals in which the aryl and alkyl radicals are as defined above such as for example benzyl, homobenzyl or phenethyl. The term arylalkoxy preferably designates the radicals in which the aryl and alkoxy radicals are as defined above such as for example benzyloxy or phenylethoxy.

The expression heteroaryl designates an aromatic radical, constituted by a ring or by 2 to 3 condensed rings, with at least one ring containing one or more identical or different heteroatoms chosen from sulphur, nitrogen or oxygen. As examples of a heteroaryl radical, there may be mentioned the pyrrolyl, imidazolyl, pyrazolyl, isothiazolyl, thiazolyl, isoxazolyl, oxazolyl, triazolyl, thiadiazolyl, pyridyl, pyrazinyl, pyrimidyl, quinolyl, isoquinolyl, quinoxalinyl, indolyl, benzoxadiazoyl, carbazolyl, purinyl, triazinyl, pyrrazolo-pyrimidyl but also thienyl, benzothienyl, furyl, benzofuryl or pyranyl, and preferably thienyl, furannyl, pyrrolyl, pyrazolyl, triazolyl, imidazolyl, thiazolyl, oxazolyl and pyridyl radicals.

In the present application, the symbol -> correspond to the attachment point of the radical.

A subject of the present invention is also compounds of general formula I as defined above, or one of its diastereoisomers as well as one of its pharmaceutically acceptable salts in which

R¹ or R² represents independently a hydrogen atom, an aryl radical or a heteroaryl radical, optionally substituted 1 to 3 times (and preferably 1 to 2 times) by a halogen atom, by an alkyl radical or by an alkoxy radical;
it being understood that R¹ and R² together can also form a ring or a heterocycle;
R³ represents a C₁ to C₈ alkyl radical or a cycloalkylalkyl radical or an aryl or arylalkyl radical;
R⁴ represents a hydrogen atom or a —CO—O—R⁵ radical with R⁵ being either a linear or branched alkyl radical or a methylfluorene or benzyl radical;
n represents the integer 1 or 2;
X represents a sulphur atom or a selenium atom;
Z represents a hydrogen atom or a radical of general formula below

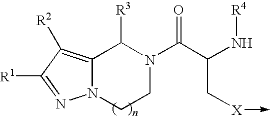

When it is stated that a radical is optionally substituted 1 to 3 times, it is preferably optionally substituted 1 to 2 times and more preferentially optionally substituted once.

The invention preferably relates to compounds of formula I as defined above and characterized in that Z represents a hydrogen atom; or a pharmaceutically acceptable salt of the latter.

Preferably also, the invention relates to compounds of formula I as defined above and characterized in that Z represents a radical of general formula

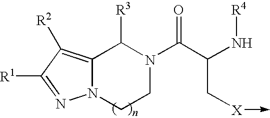

or a pharmaceutically acceptable salt of the latter.

Preferably also, the invention relates to compounds of formula I as defined above and characterized in that X represents a sulphur atom; or a pharmaceutically acceptable salt of the latter.

Preferably also, the invention relates to compounds of formula I as defined above and characterized in that X represents a selenium atom; or a pharmaceutically acceptable salt of the latter.

Preferably also, the invention relates to compounds of formula I as defined above and characterized in that R² represents a hydrogen atom; or a pharmaceutically acceptable salt of the latter.

Preferably also, the invention relates to compounds of formula I as defined above and characterized in that R⁴ represents a hydrogen atom or a radical of formula —CO—O—R⁵ and R⁵ represents an alkyl radical; or a pharmaceutically acceptable salt of the latter.

Preferably, the compound according to the invention of general formula (I) possesses an R⁴ radical which represents a hydrogen atom.

Preferably also, the invention relates to compounds of formula I as defined above and characterized in that n is equal to 1; or a pharmaceutically acceptable salt of the latter.

Preferably also, the invention relates to compounds of formula I as defined above and characterized in that R¹ represents an aryl or heteroaryl radical, the aryl radical being optionally substituted by one or more identical or different substituents chosen from halo and alkoxy,

or a radical of formula

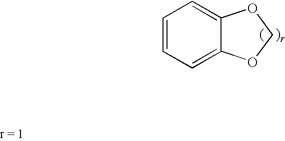

or a pharmaceutically acceptable salt of the latter.

Preferably, the invention relates to compounds of general formula (I) characterized in that R¹ represents a carbocyclic aryl radical or a heteroaryl radical, optionally substituted 1 to 3 times (and preferably 1 to 2 times) by a halogen atom, by an alkyl radical or by an alkoxy radical.

Preferably also, the invention relates to compounds of formula I as defined above and characterized in that R³ represents a C₄-C₈ alkyl, arylalkyl or cycloalkylalkyl radical; or a pharmaceutically acceptable salt of the latter.

Preferably also, the invention relates to compounds of formula I as defined above and characterized in that R² and R⁴ represent a hydrogen atom; or a pharmaceutically acceptable salt of the latter.

Preferably also, the invention relates to compounds of formula I as defined above and characterized in that R³ represents a cycloalkylalkyl or arylalkyl radical; or a pharmaceutically acceptable salt of the latter.

Preferably also, the invention relates to compounds of formula I as defined above and characterized in that R¹ represents an aryl or heteroaryl radical, the aryl radical being optionally substituted by one or more identical or different halo substituents; or a pharmaceutically acceptable salt of the latter.

Preferably also, the invention relates to compounds of formula I as defined above and characterized in that R¹ represents a heteroaryl radical; or a pharmaceutically acceptable salt of the latter.

Preferably also, the invention relates to compounds of formula I as defined above and characterized in that they comprise at least one of the following characteristics:
the cycloalkyl radical of the cycloalkyl and cycloalkylalkyl groups, is the hexyl radical;
the aryl radical of the aryl and arylalkyl groups, is the phenyl radical; and
the heteroaryl is chosen from the following radicals; furyl, thienyl, pyridinyl; or a pharmaceutically acceptable salt of the latter.

In particular, the invention relates to a compound of general formula (I) or one of its salts, chosen from the following compounds:
tert-butyl {(1R)-1-[({(2R)-2-[(tert-butoxycarbonyl)amino]-3-[(4RS)-4-(cyclohexylmethyl)-2-phenyl-6,7-dihydropyrazolo[1,5-a]pyrazin-5(4H)-yl]-3-oxopropyl}dithio)methyl]-2-[(4RS)-4-(cyclohexylmethyl)-2-phenyl-6,7-dihydropyrazolo[1,5-a]pyrazin-5(4H)-yl]-2-oxoethyl}carbamate;
tert-butyl {(1R)-1-[({(2R)-2-[(tert-butoxycarbonyl)amino]-3-oxo-3-[(4RS)-2-phenyl-4-(2-phenylethyl)-6,7-dihydropyrazolo[1,5-a]pyrazin-5(4H)-yl]propyl}dithio)methyl]-2-oxo-2-[(4RS)-2-phenyl-4-(2-phenylethyl)-6,7-dihydropyrazolo[1,5-a]pyrazin-5(4H)-yl]ethyl}carbamate;
tert-butyl {(1R)-1-[({(2R)-2-[(tert-butoxycarbonyl)amino]-3-oxo-3-[(4RS)-4-(2-phenylethyl)-2-pyridin-4-yl-6,7-dihydropyrazolo[1,5-a]pyrazin-5(4H)-yl]propyl}dithio)methyl]-2-oxo-2-[(4RS)-4-(2-phenylethyl)-2-pyridin-4-yl-6,7-dihydropyrazolo[1,5-a]pyrazin-5(4H)-yl]ethyl}carbamate;
tert-butyl {(1R)-1-[({(2R)-2-[(tert-butoxycarbonyl)amino]-3-oxo-3-[(4RS)-4-(2-phenylethyl)-2-pyridin-3-yl-6,7-dihydropyrazolo[1,5-a]pyrazin-5(4H)-yl]propyl}dithio)methyl]-2-oxo-2-[(4RS)-4-(2-phenylethyl)-2-pyridin-3-yl-6,7-dihydropyrazolo[1,5-a]pyrazin-5(4H)-yl]ethyl}carbamate;
tert-butyl {(1R)-2-[(4RS)-2-(1,3-benzodioxol-5-yl)-4-(2-phenylethyl)-6,7-dihydropyrazolo[1,5-a]pyrazin-5(4H)-yl]-1-[({(2R)-3-[(4RS)-2-(1,3-benzodioxol-5-yl)-4-(2-phenylethyl)-6,7-dihydropyrazolo[1,5-a]pyrazin-5(4H)-yl]-2-[(tert-butoxycarbonyl)amino]-3-oxopropyl}dithio)methyl]-2-oxoethyl}carbamate;
tert-butyl {(1R)-1-[({(2R)-2-[(tert-butoxycarbonyl)amino]-3-oxo-3-[(4RS)-4-(2-phenylethyl)-2-(3,4,5-trimethoxyphenyl)-6,7-dihydropyrazolo[1,5-a]pyrazin-5(4H)-yl]propyl}dithio)methyl]-2-oxo-2-[(4RS)-4-(2-phenylethyl)-2-(3,4,5-trimethoxyphenyl)-6,7-dihydropyrazolo[1,5-a]pyrazin-5(4H)-yl]ethyl}carbamate;
tert-butyl {(1R)-1-[({(2R)-2-[(tert-butoxycarbonyl)amino]-3-oxo-3-[(4RS)-4-(2-phenylethyl)-2-(2-thienyl)-6,7-dihydropyrazolo[1,5-a]pyrazin-5(4H)-yl]propyl}dithio)methyl]-2-oxo-2-[(4RS)-4-(2-phenylethyl)-2-(2-thienyl)-6,7-dihydropyrazolo[1,5-a]pyrazin-5(4H)-yl]ethyl}carbamate;
tert-butyl {(1R)-1-[({(2R)-2-[(tert-butoxycarbonyl)amino]-3-[(4RS)-2-(2-furyl)-4-(2-phenylethyl)-6,7-dihydropyrazolo[1,5-a]pyrazin-5(4H)-yl]-3-oxopropyl}dithio)methyl]-2-[(4RS)-2-(2-furyl)-4-(2-phenylethyl)-6,7-dihydropyrazolo[1,5-a]pyrazin-5(4H)-yl]-2-oxoethyl}carbamate;
tert-butyl {(1R)-1-[({(2R)-2-[(tert-butoxycarbonyl)amino]-3-oxo-3-[(4RS)-4-pentyl-2-phenyl-6,7-dihydropyrazolo[1,5-a]pyrazin-5(4H)-yl]propyl}dithio)methyl]-2-oxo-2-[(4RS)-4-pentyl-2-phenyl-6,7-dihydropyrazolo[1,5-a]pyrazin-5(4H)-yl]ethyl}carbamate;
tert-butyl {(1R)-1-[({(2R)-2-[(tert-butoxycarbonyl)amino]-3-[(4RS)-4-butyl-2-phenyl-6,7-dihydropyrazolo[1,5-a]pyrazin-5(4H)-yl]-3-oxopropyl}dithio)methyl]-2-[(4RS)-4-butyl-2-phenyl-6,7-dihydropyrazolo[1,5-a]pyrazin-5(4H)-yl]-2-oxoethyl}carbamate;
tert-butyl {(1R)-1-[({(2R)-2-[(tert-butoxycarbonyl)amino]-3-[(4RS)-2-(2,4-dichlorophenyl)-4-(2-phenylethyl)-6,7-dihydropyrazolo[1,5-a]pyrazin-5(4H)-yl]-3-oxopropyl}dithio)methyl]-2-[(4RS)-2-(2,4-dichlorophenyl)-4-(2-phenylethyl)-6,7-dihydropyrazolo[1,5-a]pyrazin-5(4H)-yl]-2-oxoethyl}carbamate;
(2R)-3-({(2R)-2-amino-3-[(4RS)-4-(cyclohexylmethyl)-2-phenyl-6,7-dihydropyrazolo[1,5-a]pyrazin-5(4H)-yl]-3-oxopropyl}dithio)-1-[(4RS)-4-(cyclohexylmethyl)-2-phenyl-6,7-dihydropyrazolo[1,5-a]pyrazin-5(4H)-yl]-1-oxopropan-2-amine hydrochloride;
(2R)-3-({(2R)-2-amino-3-oxo-3-[(4RS)-2-phenyl-4-(2-phenylethyl)-6,7-dihydropyrazolo[1,5-a]pyrazin-5(4H)-yl]propyl}dithio)-1-oxo-1-[(4RS)-2-phenyl-4-(2-phenylethyl)-6,7-dihydropyrazolo[1,5-a]pyrazin-5(4H)-yl]propan-2-amine hydrochloride;
(2R)-3-({(2R)-2-amino-3-oxo-3-[(4RS)-4-(2-phenylethyl)-2-pyridin-4-yl-6,7-dihydropyrazolo[1,5-a]pyrazin-5(4H)-yl]propyl}dithio)-1-oxo-1-[(4RS)-4-(2-phenylethyl)-2-pyridin-4-yl-6,7-dihydropyrazolo[1,5-a]pyrazin-5(4H)-yl]propan-2-amine hydrochloride;
(2R)-3-({(2R)-2-amino-3-oxo-3-[(4RS)-4-(2-phenylethyl)-2-pyridin-3-yl-6,7-dihydropyrazolo[1,5-a]pyrazin-5(4H)-yl]propyl}dithio)-1-oxo-1-[(4RS)-4-(2-phenylethyl)-2-pyridin-3-yl-6,7-dihydropyrazolo[1,5-a]pyrazin-5(4H)-yl]propan-2-amine hydrochloride;
(2R)-3-({(2R)-2-amino-3-[(4RS)-2-(1,3-benzodioxol-5-yl)-4-(2-phenylethyl)-6,7-dihydropyrazolo[1,5-a]pyrazin-5(4H)-yl]-3-oxopropyl}dithio)-1-[(4RS)-2-(1,3-benzodioxol-5-yl)-4-(2-phenylethyl)-6,7-dihydropyrazolo[1,5-a]pyrazin-5(4H)-yl]-1-oxopropan-2-amine hydrochloride;
(2R)-3-({(2R)-2-amino-3-oxo-3-[(4RS)-4-(2-phenylethyl)-2-(3,4,5-trimethoxyphenyl)-6,7-dihydropyrazolo[1,5-a]pyrazin-5(4H)-yl]propyl}dithio)-1-oxo-1-[(4RS)-4-(2-phenylethyl)-2-(3,4,5-trimethoxyphenyl)-6,7-dihydropyrazolo[1,5-a]pyrazin-5(4H)-yl]propan-2-amine hydrochloride;
(2R)-3-({(2R)-2-amino-3-oxo-3-[(4RS)-4-(2-phenylethyl)-2-(2-thienyl)-6,7-dihydropyrazolo[1,5-a]pyrazin-5(4H)-yl]propyl}dithio)-1-oxo-1-[(4RS)-4-(2-phenylethyl)-2-(2-thienyl)-6,7-dihydropyrazolo[1,5-a]pyrazin-5(4H)-yl]propan-2-amine hydrochloride
(2R)-3-({(2R)-2-amino-3-[(4RS)-2-(2-furyl)-4-(2-phenylethyl)-6,7-dihydropyrazolo[1,5-a]pyrazin-5(4H)-yl]-3-oxopropyl}dithio)-1-[(4RS)-2-(2-furyl)-4-(2-phenylethyl)-6,7-dihydropyrazolo[1,5-a]pyrazin-5(4H)-yl]-1-oxopropan-2-amine hydrochloride;
(2R)-3-({(2R)-2-amino-3-oxo-3-[(4RS)-4-pentyl-2-phenyl-6,7-dihydropyrazolo[1,5-a]pyrazin-5(4H)-yl]propyl}dithio)-1-oxo-1-[(4RS)-4-pentyl-2-phenyl-6,7-dihydropyrazolo[1,5-a]pyrazin-5(4H)-yl]propan-2-amine hydrochloride;
(2R)-3-({(2R)-2-amino-3-[(4RS)-4-butyl-2-phenyl-6,7-dihydropyrazolo[1,5-a]pyrazin-5(4H)-yl]-3-oxopropyl}dithio)-1-[(4RS)-4-butyl-2-phenyl-6,7-dihydropyrazolo[1,5-a]pyrazin-5(4H)-yl]-1-oxopropan-2-amine hydrochloride;
(2R)-3-({(2R)-2-amino-3-[(4RS)-2-(2,4-dichlorophenyl)-4-(2-phenylethyl)-6,7-dihydropyrazolo[1,5-a]pyrazin-5(4H)-yl]-3-oxopropyl}dithio)-1-[(4RS)-2-(2,4-dichlorophenyl)-4-(2-phenylethyl)-6,7-dihydropyrazolo[1,5-a]pyrazin-5(4H)-yl]-1-oxopropan-2-amine hydrochloride;
tert-butyl {(1R)-1-[({(2R)-2-[(tert-butoxycarbonyl)amino]-3-[(4S)-4-(cyclohexylmethyl)-2-phenyl-6,7-dihydropyrazolo[1,5-a]pyrazin-5(4H)-yl]-3-oxopropyl}dithio)methyl]-2-[(4S)-4-(cyclohexylmethyl)-2-phenyl-6,7-dihydropyrazolo[1,5-a]pyrazin-5(4H)-yl]-2-oxoethyl}carbamate;
(2R)-3-({(2R)-2-amino-3-[(4S)-4-(cyclohexylmethyl)-2-phenyl-6,7-dihydropyrazolo[1,5-a]pyrazin-5(4H)-yl]-3-oxopropyl}dithio)-1-[(4S)-4-(cyclohexylmethyl)-2-phenyl-6,7-dihydropyrazolo[1,5-a]pyrazin-5(4H)-yl]-1-oxopropan-2-amine hydrochloride;
tert-butyl {(1R)-1-[({(2R)-2-[(tert-butoxycarbonyl)amino]-3-[(4R)-4-(cyclohexylmethyl)-2-phenyl-6,7-dihydropyrazolo[1,5-a]pyrazin-5(4H)-yl]-3-oxopropyl}dithio)methyl]-2-[(4R)-4-(cyclohexylmethyl)-2-phenyl-6,7-dihydropyrazolo[1,5-a]pyrazin-5(4H)-yl]-2-oxoethyl}carbamate
(1R)-3-({(2R)-2-amino-3-[(4R)-4-(cyclohexylmethyl)-2-phenyl-6,7-dihydropyrazolo[1,5-a]pyrazin-5(4H)-yl]-3-oxopropyl}dithio)-1-[(4R)-4-(cyclohexylmethyl)-2-phenyl-6,7-dihydropyrazolo[1,5-a]pyrazin-5(4H)-yl]-1-oxopropan-2-amine hydrochloride
tert-butyl {(1R)-1-[({(2R)-2-[(tert-butoxycarbonyl)amino]-3-[(4RS)-4-(cyclohexylmethyl)-2-phenyl-7,8-dihydro-4H-pyrazolo[1,5-a][1,4]diazepin-5(6H)-yl]-3-oxopropyl}dithio)methyl]-2-[(4RS)-4-(cyclohexylmethyl)-2-phenyl-7,8-dihydro-4H-pyrazolo[1,5-a][1,4]diazepin-5(6H)-yl]-2-oxoethyl}carbamate
{(1R)-1-[({(2R)-2-amino-3-[(4RS)-4-(cyclohexylmethyl)-2-phenyl-7,8-dihydro-4H-pyrazolo[1,5-a][1,4]diazepin-5(6H)-yl]-3-oxopropyl}dithio)methyl]-2-[(4RS)-4-(cyclohexylmethyl)-2-phenyl-7,8-dihydro-4H-pyrazolo[1,5-a][1,4]diazepin-5(6H)-yl]-2-oxoethyl}amine hydrochloride
(2R)-2-amino-3-[(4RS)-2-(2-furyl)-4-(2-phenylethyl)-6,7-dihydropyrazolo[1,5-a]pyrazin-5(4H)-yl]-3-oxopropane-1-thiol hydrochloride
tert-butyl {(1R)-1-[({(2R)-2-[(tert-butoxycarbonyl)amino]-3-[(4RS)-4-(cyclohexylmethyl)-2-phenyl-6,7-dihydropyrazolo[1,5-a]pyrazin-5(4H)-yl]-3-oxopropyl}diselanyl)methyl]-2-[(4RS)-4-(cyclohexylmethyl)-2-phenyl-6,7-dihydropyrazolo[1,5-a]pyrazin-5(4H)-yl]-2-oxoethyl}carbamate
{(1R)-1-[({(2R)-2-amino-3-[(4RS)-4-(cyclohexylmethyl)-2-phenyl-6,7-dihydropyrazolo[1,5-a]pyrazin-5(4H)-yl]-3-oxopropyl}diselanyl)methyl]-2-[(4RS)-4-(cyclohexylmethyl)-2-phenyl-6,7-dihydropyrazolo[1,5-a]pyrazin-5(4H)-yl]-2-oxoethyl}amine hydrochloride;
(2R)-3-({(2R)-2-amino-3-oxo-3-[(4RS)-4-(2-phenylethyl)-2-pyridin-2-yl-6,7-dihydropyrazolo[1,5-a]pyrazin-5(4H)-yl]propyl}dithio)-1-oxo-1-[(4RS)-4-(2-phenylethyl)-2-pyridin-2-yl-6,7-dihydropyrazolo[1,5-a]pyrazin-5(4H)-yl]propan-2-amine hydrochloride.

The invention relates more particularly to a compound of general formula (I) chosen from the following compounds:
(2R)-3-({(2R)-2-amino-3-[(4RS)-4-(cyclohexylmethyl)-2-phenyl-6,7-dihydropyrazolo[1,5-a]pyrazin-5(4H)-yl]-3-oxopropyl}dithio)-1-[(4RS)-4-(cyclohexylmethyl)-2-phenyl-6,7-dihydropyrazolo[1,5-a]pyrazin-5(4H)-yl]-1-oxopropan-2-amine hydrochloride;
(2R)-3-({(2R)-2-amino-3-oxo-3-[(4RS)-2-phenyl-4-(2-phenylethyl)-6,7-dihydropyrazolo[1,5-a]pyrazin-5(4H)-yl]propyl}dithio)-1-oxo-1-[(4RS)-2-phenyl-4-(2-phenylethyl)-6,7-dihydropyrazolo[1,5-a]pyrazin-5(4H)-yl]propan-2-amine hydrochloride;
(2R)-3-({(2R)-2-amino-3-oxo-3-[(4RS)-4-(2-phenylethyl)-2-(2-thienyl)-6,7-dihydropyrazolo[1,5-a]pyrazin-5(4H)-yl]propyl}dithio)-1-oxo-1-[(4RS)-4-(2-phenylethyl)-2-(2-thienyl)-6,7-dihydropyrazolo[1,5-a]pyrazin-5(4H)-yl]propan-2-amine hydrochloride;
(2R)-3-({(2R)-2-amino-3-[(4RS)-2-(2,4-dichlorophenyl)-4-(2-phenylethyl)-6,7-dihydropyrazolo[1,5-a]pyrazin-5(4H)-yl]-3-oxopropyl}dithio)-1-[(4RS)-2-(2,4-dichlorophenyl)-4-(2-phenylethyl)-6,7-dihydropyrazolo[1,5-a]pyrazin-5(4H)-yl]-1-oxopropan-2-amine hydrochloride;
tert-butyl {(1R)-1-[({(2R)-2-[(tert-butoxycarbonyl)amino]-3-[(4R)-4-(cyclohexylmethyl)-2-phenyl-6,7-dihydropyrazolo[1,5-a]pyrazin-5(4H)-yl]-3-oxopropyl}dithio)methyl]-2-[(4R)-4-(cyclohexylmethyl)-2-phenyl-6,7-dihydropyrazolo[1,5-a]pyrazin-5(4H)-yl]-2-oxoethyl}carbamate;
(2R)-3-({(2R)-2-amino-3-oxo-3-[(4RS)-4-(2-phenylethyl)-2-pyridin-2-yl-6,7-dihydropyrazolo[1,5-a]pyrazin-5(4H)-yl]propyl}dithio)-1-oxo-1-[(4RS)-4-(2-phenylethyl)-2-pyridin-2-yl-6,7-dihydropyrazolo[1,5-a]pyrazin-5(4H)-yl]propan-2-amine hydrochloride.

The terminology used for the nomenclature of the above compounds is the IUPAC English terminology.

The compounds according to the present invention comprise asymmetrical centres. As a result, the compounds according to the present invention have several possible epimeric forms, i.e. the “R” or “S” configurations of said asymmetrical centres. The present invention includes all the diastereoisomeric forms and any combinations of these forms, including the “RS” mixtures. For the sake of simplicity, when no specific configuration is indicated in the structural formulae, it should be understood that all the diastereoisomeric forms and mixtures thereof are represented and described.

By salt of a compound, is meant the addition salts of said compound with an organic or inorganic acid or, if appropriate, with a base, and in particular the pharmaceutically acceptable salts of said compound.

By pharmaceutically acceptable salt, is meant in particular the addition salts of inorganic acids such as hydrochloride, hydrobromide, hydroiodide, sulphate, phosphate, diphosphate and nitrate or organic acids such as acetate, maleate, fumarate, tartrate, succinate, citrate, lactate, methanesulphonate, p-toluenesulphonate, pamoate and stearate. The salts formed from bases such as sodium or potassium hydroxide also fall within the scope of the present invention, when they can be used. For other examples of pharmaceutically acceptable salts, reference can be made to “Salt selection for basic drugs”, Int. J. Pharm. (1986), 33, 201-217.

According to the definitions of the R⁴ and Z variable groups, the compounds of the present invention can be prepared according to the different procedures described below.

### A—Preparation According to Reaction Diagram A:

The compound of formula (I) according to the invention in which R⁴ represents a radical of formula —CO—O—R⁵ can be prepared according to the following diagram A:

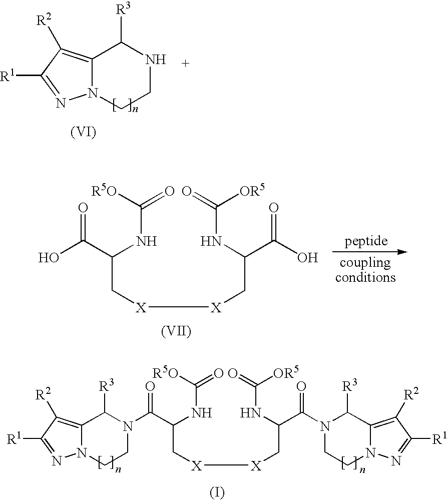

The compounds of general formula (I) in which R⁴ represents a radical of formula —CO—O—R⁵, can be prepared under so-called peptide coupling conditions (Montalbetti et al. Tetrahedron 2005, 61, 10827), by reacting a carboxylic acid of general formula (VII) (N-protected cystine or N-protected seleno-cystine) in which R⁵ is as defined above, with a compound of general formula (VI) at a temperature comprised between 0° C. and 30° C. (preferably at ambient temperature) in an aprotic solvent such as for example DCM, DCE, THF or MeCN.

### B—Preparation According to Reaction Diagram B:

The compounds of formula (I) according to the invention in which R⁴ represents a hydrogen atom can be prepared according to the following diagram B:

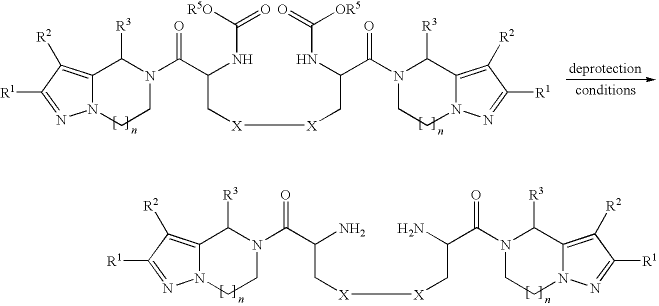

The compounds of general formula (I) in which R⁴ represents a hydrogen atom, can be prepared by treating a compound of general formula (I) in which R⁴ represents a radical of formula —CO—O—R⁵, under deprotection conditions. These conditions are, for example, an acid treatment (TFA, HCl, HCOOH) for the tert-butoxycarbonyl (Boc), a treatment with a secondary amine (piperidine) for the 9-fluorenylmethyloxycarbonyl (Fmoc) group, at a temperature comprised between 0° C. and 30° C., and preferably at ambient temperature.

### C—Preparation According to Reaction Diagram C:

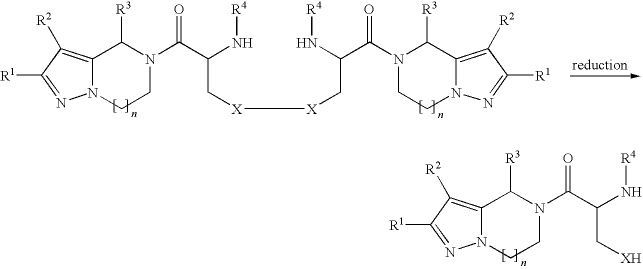

The compounds of general formula (I) in which Z represents the hydrogen atom can be prepared by treating the corresponding compounds of general formula (I) in which Z does not represent the hydrogen atom, under reduction conditions such as, for example, sodium borohydride or dithiothreitol in a protic solvent such as, for example, methanol or ethanol.

### Preparation of the Compound of Formula (VI):

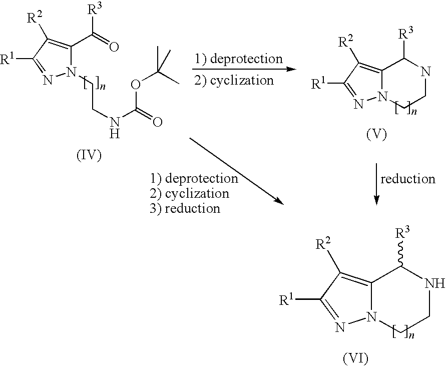

The compounds of formula (VI) can be obtained from the compound of formula (IV) either indirectly via the synthesis of intermediate compound (V) or directly.

The compounds of general formula (V) can be prepared by treating a compound of general formula (IV) under acid conditions in order to remove the Boc protective group, then by adding a base in order to neutralize the acidity and promote the condensation of the free amine with the carbonyl radical bearing the R³ radical. The deprotection is carried out for example in a mixture of TFA and DCM, or also in formic acid, at a temperature comprised between 0° C. and 30° C., preferably at ambient temperature. The neutralization can be obtained, for example, by adding TEA to the reaction medium.

The compounds of general formula (VI) can be obtained by reducing to amine the imine function of the compounds of general formula (V). This reaction is generally carried out with sodium borohydride in MeOH or EtOH at a temperature comprised between 0° C. and 30° C. This reaction can also be carried out under hydrogenation conditions by asymmetric transfer, in such a way that compound (VI) is obtained with a high enantiomeric excess. An example of such a conversion is described by Williams G D et al. Org. Lett. 2003, 5, 4227.

These same compounds of general formula (VI), both racemic and with a high enantiomeric excess, can also be prepared from a compound of general formula (IV) by successively carrying out the stages of deprotection in order to release the amine function, of condensation and of reduction in the same reactor and without purifying the intermediate products.

The deprotection of compound (IV) can be obtained by treatment with an acid such as for example trifluoroacetic acid or formic acid, without solvent or in a solvent such as, for example, dichloromethane, THF or acetonitrile, at a temperature comprised between 0° C. and 50° C. and preferably at ambient temperature. The conditions for formation of the imine (V) and the reduction to amine (VI) are known to a person skilled in the art by the term reductive amination and can be achieved in various ways such as for example sodium cyanoborohydride in acetonitrile, sodium triacetoxyborohydride, or also, for the cyclic imines such as the compounds of formula (V), sodium borohydride in methanol. When the reductive amination is carried out starting from a ketone as in the compounds of formula (V), there is formation of a chiral centre and it is then useful for the reduction of the imine to be carried out in favour of one of the two possible epimers relative to this chiral centre. Such a conversion of the imines to amines can be obtained under so-called hydrogenation conditions by asymmetric transfer. The source of hydrogen is then preferably formic acid or one of its salts such as, for example, sodium formate, the solvent can be formic acid in the presence of a base such as, for example, triethylamine. The reaction is catalyzed by a ruthenium complex obtained by reaction between bis((η⁶-p-cymene)dichlororuthenium) and a tosylated asymmetric diamine as chiral auxiliary such as, for example, (1R,2R)—N-(p-toluenesulphonyl)-1,2-diphenylethylenediamine ((R,R)-TsDPEN). Examples of such catalysts used for the hydrogenation by asymmetric transfer of cyclic imines are described in: Org Lett 2003, vol 5, pp 4227-4230; Green Chem 2007, vol 9, pp 23-25; Green Chem 2007, vol 9, pp 391-397; Chem Commun 2007, pp 1825-1827.

### Preparation of the Intermediate of Formula (IV):

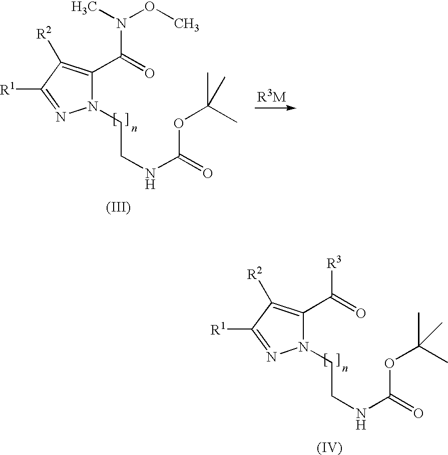

The compounds of general formula (IV) can be prepared by reacting a compound of general formula (III) with an organometallic reagent of general formula R³M in which R³ is as defined above and M represents for example Li or Mg (MgBr or MgCl), these reagents being able to be of commercial origin or produced in situ according to methods known to a person skilled in the art. This reaction is carried out in an aprotic solvent such as for example THF, at a temperature comprised between −80° C. and 0° C. for the organolithium compounds and between 0° C. and 60° C., and preferably at ambient temperature for the organomagnesium compounds.

### Preparation of the Intermediate of Formula (III):

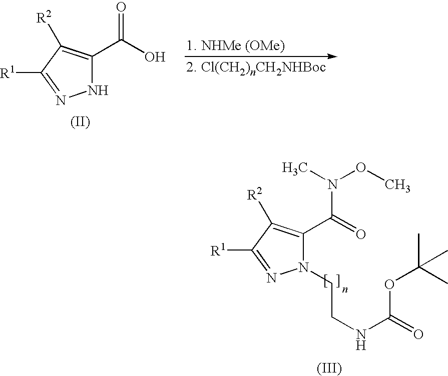

The derivatives of general formula (III) can be prepared under so-called peptide coupling conditions (Montalbetti et al. Tetrahedron 2005, 61, 10827), by reacting carboxylic acid (II) with N,O-dimethylhydroxylamine, at a temperature comprised between 0° C. and 100° C. (preferably at ambient temperature), in an inert solvent such as for example dichloromethane (DCM), THF or also DMF. The intermediate thus obtained can then be N-alkylated with tert-butyl (2-chloroethyl)carbamate or tert-butyl (3-bromopropyl)carbamate in the presence of a base such as sodium carbonate or potassium tert-butoxide, optionally combined with a phase transfer agent such as tetrabutylammonium bromide, at a temperature comprised between ambient temperature and 110° C. and in an aprotic solvent, such as, for example, at 60° C. in THF, at 80° C. in MeCN or also at 110° C. in DMF.

The carboxylic acids (II) are generally commercial products or can be prepared by standard methods known to a person skilled in the art.

A subject of the invention is also a process for the preparation of a compound of formula (I) as defined above, characterized in that a compound of formula (VI)

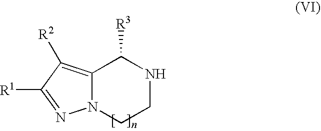

in which the radicals R¹, R² and R³ and n are as defined above, is reacted with a carboxylic acid of general formula (VII)

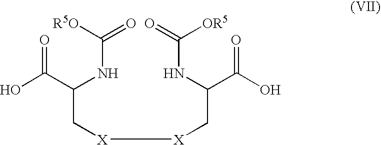

in which R⁵ and X are as defined above, under so-called peptide coupling conditions, at a temperature comprised between 0° C. and 30° C., in an aprotic solvent, in order to obtain the compound of formula (I) in which Z is different from the hydrogen atom and R⁴ represents the —CO—O—R⁵ radical,
compound of formula (I) in which R⁴ represents the —CO—O—R⁵ radical which can be deprotected, in order to obtain the compound of formula (I) in which Z is different from the hydrogen atom and R⁴ represents the hydrogen atom,
and finally the compound of formula (I) in which Z is different from the hydrogen atom, can be reduced in order to obtain the corresponding compound of formula (I) in which Z represents the hydrogen atom.

A subject of the invention is also a compound of general formula (VI)

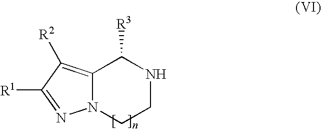

in racemic, enantiomeric form or any combinations of these forms, in which the R¹, R² and R³ radicals and n are as defined above.

A subject of the invention is also a process for the preparation of a compound of formula (VI) as defined above, characterized in that compound (IV)

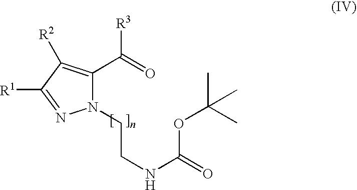

in which the R¹, R² and R³ radicals and n are as defined above,
is subjected to acid conditions in order to release the amine function and form, after neutralization, compound (V)

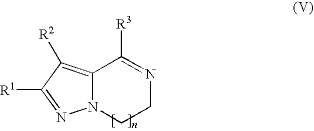

in which the R¹, R² and R³ radicals and n are as defined above, then the imine function of the compound of general formula (V) thus formed is then subjected to reducing conditions in order to produce the corresponding cyclic amine (VI).

A subject of the present invention is also a compound of general formula (I) as defined above or a pharmaceutically acceptable salt of such a compound, for its use as a therapeutically active ingredient.

A subject of the present invention is also a pharmaceutical composition comprising, as active ingredient, a compound of general formula (I) as defined above, or a pharmaceutically acceptable salt of such a compound, with at least one pharmaceutically acceptable excipient.

A subject of the present invention is also, as a medicament, a compound of general formula (I) as defined above, or a pharmaceutically acceptable salt of such a compound.

A subject of the present invention is also the use of at least one compound of general formula (I) as defined above or one of the pharmaceutically acceptable salts of such a compound, for preparing a medicament intended to prevent or treat a disease or a disorder chosen from the following diseases or the following disorders: cancers, non-tumorous proliferative diseases, tumorous proliferative diseases, neurodegenerative diseases, parasitic diseases, viral infections, spontaneous alopecia, alopecia induced by exogenous products, radiation-induced alopecia, auto-immune diseases, graft rejections, inflammatory diseases, allergies or pain.

A subject of the present invention is preferentially the use of at least one compound of general formula (I) as defined above or one of the pharmaceutically acceptable salts of such a compound, for preparing a medicament intended to treat or prevent cancers,

and very preferentially cancers of the colon, rectum, stomach, lungs, pancreas, kidney, testicles, breast, uterus, ovary, prostate, skin, bone, spinal cord, neck, tongue, head as well as sarcomas, carcinomas, fibroadenomas, neuroblastomas, leukemias, melanomas.

The compound of general formula (I) or its salt used according to the invention or the combination according to the invention can be in the form of a solid, for example powders, granules, tablets, gelatin capsules, liposomes or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax.

The compound of general formula (I) or its salt used according to the invention or the combination according to the invention can also be presented in liquid form, for example, solutions, emulsions, suspensions or syrups. Appropriate liquid supports can be, for example, water, organic solvents such as glycerol or the glycols, as well as mixtures thereof, in varying proportions, in water.

The administration of a compound of general formula (I) or its salt used according to the invention or the combination according to the invention can be carried out by topical, oral, parenteral route, by intramuscular, sub-cutaneous injection.

The dose of a product according to the present invention, to be provided for the treatment of the abovementioned diseases or disorders, varies according to the method of administration, the age and body weight of the subject to be treated as well as the state of the latter, and will be finally decided by the attending doctor or vet. Such a quantity determined by the attending doctor or vet is here called the “therapeutically effective quantity”.

By way of example, the administration dose envisaged for a medicament according to the invention is comprised between 0.1 mg and 10 g according to the type of active compound used.

All the technical and scientific terms used in the present text have the meaning known to a person skilled in the art. Moreover, all the patents (or patent applications) as well as the other bibliographical references are incorporated by way of reference.

### Experimental Part

According to the above definitions of the R1, R2, R3, R4, R5, X and Z variable groups, the compounds of the invention can be prepared according to the different procedures described above.

The examples below are presented in order to illustrate the above procedures and should in no event be considered as a limit to the scope of the invention.

### EXAMPLE 1

### tert-butyl {(1R)-1-[({(2R)-2-[(tert-butoxycarbonyl)amino]-3-[(4RS)-4-(cyclohexylmethyl)-2-phenyl-6,7-dihydropyrazolo[1,5-a]pyrazin-5(4H)-yl]-3-oxopropyl}dithio)methyl]-2-[(4RS)-4-(cyclohexylmethyl)-2-phenyl-6,7-dihydropyrazolo[1,5-a]pyrazin-5(4H)-yl]-2-oxoethyl}carbamate

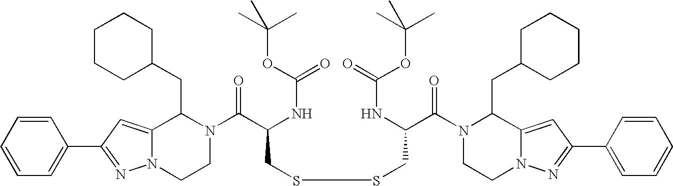

### 1a. N-methoxy-N-methyl-3-phenyl-1H-pyrazole-5-carboxamide

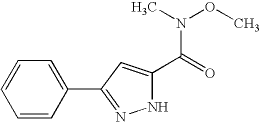

3-(phenyl)-1H-pyrazole-5-carboxylic acid (7.53 g, 40 mmol) in solution in DCM (200 mL) is reacted with N,O-dimethylhydroxylamine hydrochloride, (7.80 g, 80 mmol), triethylamine (22.3 mL, 160 mmol, 4 eq.) and 1-(3-dimethylaminopropyl)-3-ethylcarbodiimide hydrochloride (15.4 g, 80 mmol, 2 eq.). The reaction medium is stirred at ambient temperature until the starting product disappears (24 hours; TLC, eluent: DCM/MeOH=90/10). The volatile compounds are then evaporated off and the residue purified on SiO₂ (eluent: DCM/MeOH=99/1) in order to obtain the compound of Example 1a (6.22 g, 67%) in the form of a beige powder.

NMR-¹H (δ ppm, DMSO): 3.32 (s, 3H); 3.78 (s, 3H); 7.18 (s, 1H); 7.34 (s, 1H); 7.43 (s, 2H); 7.86 (s, 2H); 13.66 (s, 1H).

NMR-¹³C (δ ppm, DMSO): 32.47; 61.45; 104.94; 125.19; 127.67; 128.65; 133.06; 135.17; 151.04; 158.63.

MH⁺ experimental=232.21; M theoretical=231.25

Melting point: 135-138° C.

### 1b. tert-butyl [2-(5-{[methoxy(methyl)amino]carbonyl}-3-phenyl-1H-pyrazol-1-yl)ethyl]carbamate

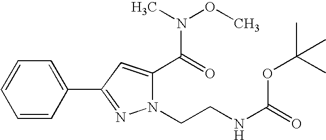

The compound of Example 1a (4.58 g, 19.8 mmol) in DMF (80 mL) is reacted with tert-butyl (2-chloroethyl)carbamate (4.27 g, 23.8 mmol, 1.2 eq.) in the presence of sodium carbonate (3.01 g, 21.8 mmol, 1.1 eq.). The reaction medium is heated at 110° C. for 5 hours and 30 minutes (TLC, eluent: DCM/MeOH=98/2). The DMF is then evaporated off and the residue dissolved in AcOEt, followed by washing twice with water. The organic phase is dried over sodium sulphate, then filtered and concentrated under vacuum. The residue is purified on SiO₂ (eluent: heptane/AcOEt=60/40) in order to obtain the compound of Example 1b (6.4 g, 86%) in the form of a translucent oil.

NMR-¹H (δ ppm, DMSO): 1.31 (s, 9H); 3.31-3.35 (m, 2H); 3.30 (s, 3H); 3.69 (s, 3H); 4.43 (t, 2H); 6.84 (br, 1H); 7.14 (s, 1H); 7.31 (s, 1H); 7.41 (s, 2H); 7.83 (s, 2H).

NMR-¹³C (δ ppm, DMSO): 27.54; 28.10; 40.39; 50.63; 61.26; 77.63; 105.57; 125.15; 127.76; 128.61; 132.56; 134.76; 148.78; 155.44; 159.51.

MH⁺ experimental=375.26; M theoretical=374.44

### 1c. tert-butyl {2-[5-(cyclohexylacetyl)-3-phenyl-1H-pyrazol-1-yl]ethyl}carbamate

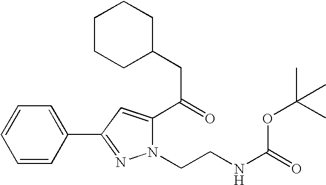

Magnesium (1.07 g, 44 mmol, 5.5 eq.) is placed under an inert atmosphere in a flask (100 mL), then iodine (2 crystals), anhydrous THF (20 mL) and bromomethylcyclohexane (560 μL, 4 mmol, 0.5 eq.) are introduced. The reaction medium is heated at 30° C. for 5 minutes in order to initiate the formation of the magnesium compound, then heating is stopped and stirring is continued for 30 minutes. The disappearance of the brown colour associated with iodine and an effervescence at the surface of the metal accompanied by the development of cloudiness in the solution are noted. Then bromomethylcyclohexane (5.02 mL, 36 mmol, 4.5 eq.) in solution in anhydrous THF (20 mL) is added dropwise (over 10 minutes), and a rise in the temperature of the medium as well as the conversion of the magnesium are noted. Once the temperature of the reaction has returned to 22° C. and almost no more magnesium remains (˜1 hour), the compound of Example 1b (3 g, 8 mmol) in solution in anhydrous THF (10 mL) is added dropwise (over 1 hour). The reaction medium which progressively becomes yellow (TLC, eluent: DCM/MeOH=98/2) is stirred for 6 hours. Water is carefully added then the mixture obtained is partitioned between ethyl acetate (100 mL) and water (100 mL). The aqueous phase is extracted with ethyl acetate and the organic phases are concentrated then washed with a saturated solution of sodium chloride under reduced pressure, dried over sodium sulphate and recombined. The residue is purified on SiO₂ (eluent: heptane/AcOEt=80/20) in order to obtain the compound of Example 1c (1.62 g, 49%) in the form of a white solid.

NMR-¹H (δ ppm, DMSO): 0.97-1.72 (m, 10H); 1.28 (s, 9H); 1.85-1.89 (m, 1H); 2.80 (d, 2H); 3.29 (q, 2H); 4.52 (t, 2H); 6.86 (br, 1H); 7.33 (t, 1H); 7.42 (t, 2H); 7.66 (s, 1H); 7.85 (s, 2H).

NMR-¹³C (δ ppm, DMSO): 25.82; 25.98; 28.29; 32.71; 34.28; 47.58; 51.53; 77.73; 109.56; 125.30; 128.08; 128.84; 132.52; 140.25; 148.93; 155.61; 191.54.

MH⁺ experimental=412.27; M theoretical=411.54

### 1d. 4-(cyclohexylmethyl)-2-phenyl-6,7-dihydropyrazolo[1,5-a]pyrazine

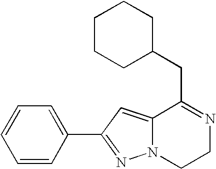

The compound of Example 1c (573 mg, 1.4 mmol) in solution in DCM (3 mL) is reacted with trifluoroacetic acid (3 mL) for 5 hours, then the reaction medium is concentrated under reduced pressure. The yellow oil obtained is dissolved in DCM (3 mL), triethylamine is added (3 mL), and the reaction medium is stirred at ambient temperature for 17 hours (TLC, eluent: DCM/MeOH=98/2). The volatile compounds are evaporated off under reduced pressure then the residue is dissolved in DCM. The residue is washed with water then with a saturated solution of sodium chloride. The organic phase is dried over sodium sulphate then concentrated under reduced pressure. The compound of Example 1d is obtained (454 mg, 100%) in the form of a yellow oil.

NMR-¹H (δ ppm, DMSO): 0.95-1.74 (m, 10H); 1.74-1.81 (m, 1H); 2.47-2.50 (m, 2H); 3.93 (t, 2H); 4.13 (t, 2H); 7.10 (s, 1H); 7.31 (t, 1H); 7.41 (t, 2H); 7.84 (s, 2H).

NMR-¹³C (δ ppm, DMSO): 26.69; 26.82; 33.57; 36.32; 43.74; 44.30; 48.40; 101.71; 125.98; 128.62; 129.57; 133.65; 140.36; 159.29.

MH⁺ experimental=294.36; M theoretical=293.41

### 1e. (4RS)-4-(cyclohexylmethyl)-2-phenyl-4,5,6,7-tetrahydropyrazolo[1,5-a]pyrazine

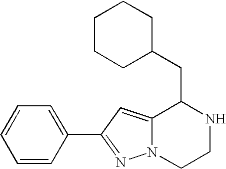

The compound of Example 1d (408 mg, 1.4 mmol) in solution in MeOH (20 mL) is reacted with sodium borohydride (63 mg, 1.7 mmol, 1.2 eq.) at ambient temperature, for 30 minutes. The volatile compounds are evaporated off (TLC, eluent: DCM/MeOH=95/5; developer: ninhydrin), then the residue is dissolved in DCM. The residue is washed with water, then with a saturated solution of sodium chloride. The organic phase is dried over sodium sulphate, then concentrated under reduced pressure. The product is purified over SiO₂ (eluent: DCM/MeOH=98/2) in order to obtain the compound of Example 1e (372 mg, 91%).

A white solid; Melting point: 128-130° C.

MH⁺ experimental=296.39; M theoretical=295.43

NMR-¹H (δ ppm, DMSO): 0.89-1.85 (m, 13H); 2.44 (br, 1H); 3.00 (ddd, 1H); 3.27 (dt, 1H); 3.91-4.00 (m, 3H); 6.46 (s, 1H); 7.25 (t, 1H); 7.36 (t, 2H); 7.75 (d, 2H).

NMR-¹³C (δ ppm, DMSO): 25.60; 25.89; 31.83; 33.15; 33.93; 41.65; 47.86; 50.06; 97.90; 125.26; 127.50; 128.88; 134.07; 144.56; 149.12.

### 1f. tert-butyl{(1R)-1-[({(2R)-2-[(tert-butoxycarbonyl)amino]-3-[(4RS)-4-(cyclohexylmethyl)-2-phenyl-6,7-dihydropyrazolo[1,5-a]pyrazin-5(4H)-yl]-3-oxopropyl}dithio)methyl]-2-[(4RS)-4-(cyclohexylmethyl)-2-phenyl-6,7-dihydropyrazolo[1,5-a]pyrazin-5(4H)-yl]-2-oxoethyl}carbamate

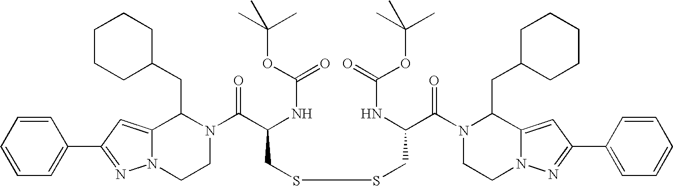

The compound of Example 1e (484 mg, 2.2 mmol, 2.1 eq.) and N,N′-di-Boc-L-cystine (463 mg 1.05 mmol, 1 eq.) are dissolved in anhydrous THF (15 mL). The medium is cooled down to a temperature comprised between 0 and 5° C.; then diisopropylethylamine (1.23 mL, 7 mmol, 6.7 eq.) and O-(7-azobenzotriazol-1-yl)-1,1,3,3-tetramethyluronium hexafluorophosphate (HATU, 798 mg, 2.1 mmol, 2 eq.) in solution in anhydrous acetonitrile (8 mL) are added. The reaction medium is stirred at ambient temperature for 18 hours (TLC, eluent: DCM/MeOH=90/10) and the volatile compounds are evaporated off. The residue is dissolved in DCM and washed with water, then twice with a saturated solution of sodium chloride. The organic phase is dried over sodium sulphate, then filtered and the volatile compounds are evaporated off under reduced pressure. The residue obtained is purified on a silica column (eluent: DCM/MeOH=95/5) in order to obtain 647 mg (73%) of the compound of Example 1f (mixture of 3 diastereoisomers) in the form of a white solid.

Melting point: 129-132° C.

[M+2H]²⁺ experimental=498.43; M theoretical=995.59

NMR-¹H (δ ppm, DMSO): 1.25-1.37 (m, 18H); 0.70-1.75 (m, 26H); 2.8-3.1 (m, 4H); 3.5-4.5 (m, 10H); 5.80 (m, 2H); 6.31-6.37 (m, 2H) 7.16-7.20 (m, 2H) 7.23-7.29 (m, 4H); 7.60-7.65 (m, 4H).

### EXAMPLE 2

### tert-butyl {(1R)-1-[({(2R)-2-[(tert-butoxycarbonyl)amino]-3-oxo-3-[(4RS)-2-phenyl-4-(2-phenylethyl)-6,7-dihydropyrazolo[1,5-a]pyrazin-5(4H)-yl]propyl}dithio)methyl]-2-oxo-2-[(4RS)-2-phenyl-4-(2-phenylethyl)-6,7-dihydropyrazolo[1,5-a]pyrazin-5(4H)-yl]ethyl}carbamate

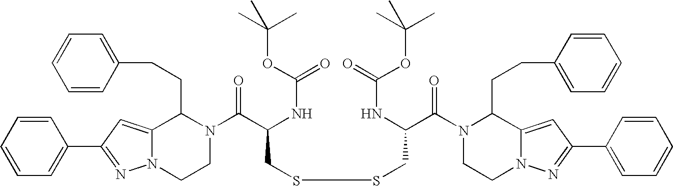

### 2a. (4RS)-2-phenyl-4-(2-phenylethyl)-4,5,6,7-tetrahydropyrazolo[1,5-a]pyrazine

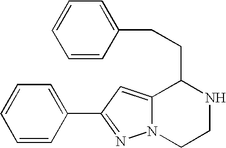

The compound below is synthesized according to a method analogous to that described in Example 1e.

Yellow oil —MH⁺ experimental=304.14; M theoretical=303.41

NMR-¹H (δ ppm, DMSO): 1.88-1.91 (m, 1H); 2.13-2.14 (m, 1H); 2.67 (br, 1H); 2.74-2.81 (m, 2H); 3.03 (ddd, 1H); 3.28-3.31 (m, 1H); 3.89 (dd, 1H); 3.97-4.03 (m, 2H); 6.55 (s, 1H); 7.16-7.19 (m, 1H); 7.23-7.31 (m, 5H); 7.36 (t, 2H); 7.76 (d, 2H).

NMR-¹³C (δ ppm, DMSO): 31.58; 36.63; 41.91; 47.72; 52.09; 97.97; 125.08; 125.89; 127.34; 128.47; 128.52; 128.69; 133.86 142.19; 143.53; 149.03.

### 2b. tert-butyl {(1R)-1-[({(2R)-2-[(tert-butoxycarbonyl)amino]-3-oxo-3-[(4RS)-2-phenyl-4-(2-phenylethyl)-6,7-dihydropyrazolo[1,5-a]pyrazin-5(4H)-yl]propyl}dithio)methyl]-2-oxo-2-[(4RS)-2-phenyl-4-(2-phenylethyl)-6,7-dihydropyrazolo[1,5-a]pyrazin-5(4H)-yl]ethyl}carbamate

The compound of Example 2 is synthesized according to a method analogous to that described in Example 1f.

White solid—Melting point: 123-127° C.

[M+2H]²⁺ experimental=506.39; M theoretical=1011.32

NMR-¹H (δ ppm, DMSO): 1.25-1.37 (m, 18H); 2.06-2.11 (m, 4H); 2.64-2.70 (m, 4H); 2.93-3.17 (m, 4H); 3.44-4.84 (m, 10H); 5.72 (m, 2H); 6.64 (m, 2H) 7.14-7.28 (m, 12H) 7.35-7.40 (m, 4H); 7.55-7.58 (m, 2H); 7.74-7.76 (m, 4H).

### EXAMPLE 3

### tert-butyl {(1R)-1-[({(2R)-2-[(tert-butoxycarbonyl)amino]-3-oxo-3-[(4RS)-4-(2-phenylethyl)-2-pyridin-4-yl-6,7-dihydropyrazolo[1,5-a]pyrazin-5(4H)-yl]propyl}dithio)methyl]-2-oxo-2-[(4RS)-4-(2-phenylethyl)-2-pyridin-4-yl-6,7-dihydropyrazolo[1,5-a]pyrazin-5(4H)-yl]ethyl}carbamate

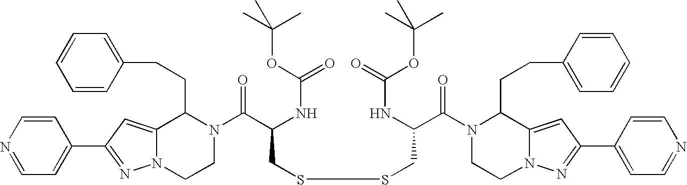

### 3a. (4RS)-4-(2-phenylethyl)-2-pyridin-4-yl-4,5,6,7-tetrahydropyrazolo[1,5-a]pyrazine

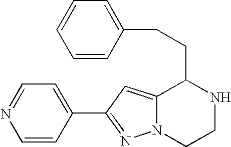

The compound below is synthesized according to a method analogous to that described in Example 1e.

Pale yellow powder—Melting point: 134-136° C.

MH⁺ experimental=305.34; M theoretical=304.39

NMR-¹H (δ ppm, DMSO): 1.89-1.91 (m, 1H); 2.13-2.15 (m, 1H); 2.74-2.81 (m, 3H); 3.04 (ddd, 1H); 3.32-3.34 (m, 1H); 3.90 (dd, 1H); 4.01-4.07 (m, 2H); 6.76 (s, 1H); 7.16-7.20 (m, 1H); 7.26-7.32 (m, 4H); 7.71 (d, 2H); 8.54 (d, 2H).

NMR-¹³C (δ ppm, DMSO): 31.59; 36.67; 41.86; 48.03; 52.12; 99.40; 119.56; 125.99; 128.58; 140.84; 142.19; 144.18; 146.72; 150.26.

### 3b. tert-butyl {(1R)-1-[({(2R)-2-[(tert-butoxycarbonyl)amino]-3-oxo-3-[(4RS)-4-(2-phenylethyl)-2-pyridin-4-yl-6,7-dihydropyrazolo[1,5-a]pyrazin-5(4H)-yl]propyl}dithio)methyl]-2-oxo-2-[(4RS)-4-(2-phenylethyl)-2-pyridin-4-yl-6,7-dihydropyrazolo[1,5-a]pyrazin-5(4H)-yl]ethyl}carbamate

The compound of Example 3 is synthesized according to a method analogous to that described in Example 1f.

White solid—Melting point: 149-152° C.

[M+2H]²⁺ experimental=507.43; M theoretical=1013.3

NMR-¹H (δ ppm, DMSO): 1.23-1.36 (m, 18H); 2.07-2.11 (m, 4H); 2.62-2.96 (m, 4H); 3.10-4.84 (m, 14H); 5.72 (m, 2H); 6.89 (m, 2H); 7.13-7.27 (m, 10H); 7.57-7.59 (m, 2H); 7.79-7.83 (m, 4H); 8.59-8.61 (m, 4H).

### EXAMPLE 4

### tert-butyl {(1R)-1-[({(2R)-2-[(tert-butoxycarbonyl)amino]-3-oxo-3-[(4RS)-4-(2-phenylethyl)-2-pyridin-3-yl-6,7-dihydropyrazolo[1,5-a]pyrazin-5(4H)-yl]propyl}dithio)methyl]-2-oxo-2-[(4RS)-4-(2-phenylethyl)-2-pyridin-3-yl-6,7-dihydropyrazolo[1,5-a]pyrazin-5(4H)-yl]ethyl}carbamate

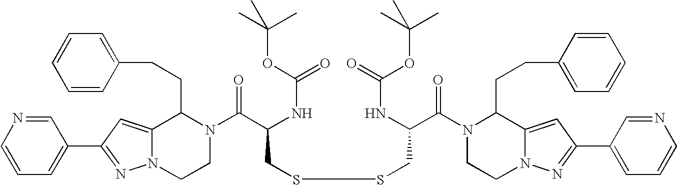

### 4a. (4RS)-4-(2-phenylethyl)-2-pyridin-3-yl-4,5,6,7-tetrahydropyrazolo[1,5-a]pyrazine

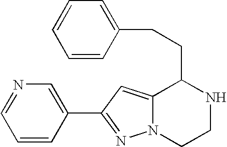

The compound below is synthesized according to a method analogous to that described in Example 1e.

Yellow powder—Melting point: 92-94° C.

MH⁺ experimental=305.30; M theoretical=304.39

NMR-¹H (δ ppm, DMSO): 1.89-1.91 (m, 1H); 2.13-2.14 (m, 1H); 2.69 (br, 1H); 2.74-2.82 (m, 2H); 3.04 (ddd, 1H); 3.29-3.33 (m, 1H); 3.90 (dd, 1H); 4.00-4.06 (m, 2H); 6.69 (s, 1H); 7.16-7.20 (m, 1H); 7.26-7.32 (m, 4H); 7.39 (dd, 1H); 8.11 (dt, 1H); 8.46 (dd, 1H); 8.97 (d, 1H).

NMR-¹³C (δ ppm, DMSO): 31.55; 36.64; 41.84; 47.84; 52.07; 98.48; 123.90; 125.91; 128.52; 129.50; 132.18; 142.15; 143.87; 146.35; 146.42; 148.39.

### 4b. tert-butyl {(1R)-1-[({(2R)-2-[(tert-butoxycarbonyl)amino]-3-oxo-3-[(4RS)-4-(2-phenylethyl)-2-pyridin-3-yl-6,7-dihydropyrazolo[1,5-a]pyrazin-5(4H)-yl]propyl}dithio)methyl]-2-oxo-2-[(4RS)-4-(2-phenylethyl)-2-pyridin-3-yl-6,7-dihydropyrazolo[1,5-a]pyrazin-5(4H)-yl]ethyl}carbamate

The compound of Example 4 is synthesized according to a method analogous to that described in Example 1f.

White solid—Melting point: 115-130° C.

[M+2H]²⁺ experimental=507.43; M theoretical=1013.3

NMR-¹H (δ ppm, DMSO): 1.35 (m, 18H); 2.06-2.11 (m, 4H); 2.64-2.68 (m, 4H); 2.82-3.23 (m, 4H); 3.88-4.90 (m, 10H); 5.72 (m, 2H); 6.78 (m, 2H); 7.14-7.27 (m, 10H); 7.40-7.43 (m, 2H); 7.60-7.61 (m, 2H); 7.79-7.83 (m, 4H); 8.09-8.12 (m, 2H); 8.48-8.49 (m, 2H); 8.97 (s, 2H).

### EXAMPLE 5

### tert-butyl {(1R)-2-[(4RS)-2-(1,3-benzodioxol-5-yl)-4-(2-phenylethyl)-6,7-dihydropyrazolo[1,5-a]pyrazin-5(4H)-yl]-1-[({(2R)-3-[(4RS)-2-(1,3-benzodioxol-5-yl)-4-(2-phenylethyl)-6,7-dihydropyrazolo[1,5-a]pyrazin-5(4H)-yl]-2-[(tert-butoxycarbonyl)amino]-3-oxopropyl}dithio)methyl]-2-oxoethyl}carbamate

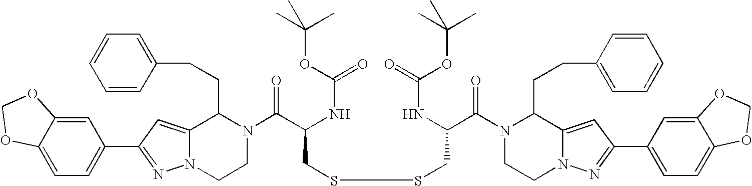

### 5a. (4RS)-2-(1,3-benzodioxol-5-yl)-4-(2-phenylethyl)-4,5,6,7-tetrahydropyrazolo[1,5-a]pyrazine

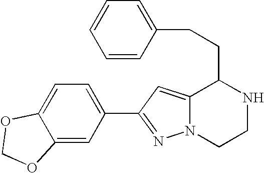

The compound below is synthesized according to a method analogous to that described in Example 1e.

Yellow oil—MH⁺ experimental=348.18; M theoretical=347.42

NMR-¹H (δ ppm, DMSO): 1.86-1.91 (m, 1H); 2.09-2.14 (m, 1H); 2.63 (br, 1H); 2.71-2.80 (m, 2H); 3.00 (ddd, 1H); 3.26-3.30 (m, 1H); 3.85 (dd, 1H); 3.93-4.01 (m, 2H); 6.00 (s, 2H); 6.47 (s, 1H); 6.89 (d, 1H); 7.19 (m, 1H); 7.24-7.31 (m, 6H).

NMR-¹³C (δ ppm, DMSO): 31.56; 36.64; 41.91; 47.63; 52.06; 97.67; 101.03; 105.51; 108.53; 118.63; 125.88; 128.26; 128.47; 142.20; 143.44; 146.63; 147.70; 148.87.

### 5b. tert-butyl {(1R)-2-[(4RS)-2-(1,3-benzodioxol-5-yl)-4-(2-phenylethyl)-6,7-dihydropyrazolo[1,5-a]pyrazin-5(4H)-yl]-1-[({(2R)-3-[(4RS)-2-(1,3-benzodioxol-5-yl)-4-(2-phenylethyl)-6,7-dihydropyrazolo[1,5-a]pyrazin-5(4H)-yl]-2-[(tert-butoxycarbonyl)amino]-3-oxopropyl}dithio)methyl]-2-oxoethyl}carbamate

The compound of Example 5 is synthesized according to a method analogous to that described in Example 1f.

Yellow paste—[M+2H]²⁺ experimental=550.30; M theoretical=1099.34

NMR-¹H (δ ppm, DMSO): 1.35 (m, 18H); 2.06-2.11 (m, 4H); 2.63-2.66 (m, 4H); 2.81-3.13 (m, 4H); 3.82-4.84 (m, 10H); 5.66-5.71 (m, 2H); 6.01-6.02 (m, 4H); 6.56 (m, 2H); 6.89-6.92 (m, 2H); 7.15-7.28 (m, 14H); 7.55-7.61 (m, 2H).

NMR-¹³C (δ ppm, DMSO): 28.27; 31.40; 32.49; 51.42, 55.05; 99.98; 101.13; 105.58; 108.60; 118.85; 125.95; 127.70; 128.40; 141.48; 141.64; 146.91; 147.76; 149.83.

### EXAMPLE 6

### tert-butyl {(1R)-1-[({(2R)-2-[(tert-butoxycarbonyl)amino]-3-oxo-3-[(4RS)-4-(2-phenylethyl)-2-(3,4,5-trimethoxyphenyl)-6,7-dihydropyrazolo[1,5-a]pyrazin-5(4H)-yl]propyl}dithio)methyl]-2-oxo-2-[(4RS)-4-(2-phenylethyl)-2-(3,4,5-trimethoxyphenyl)-6,7-dihydropyrazolo[1,5-a]pyrazin-5(4H)-yl]ethyl}carbamate

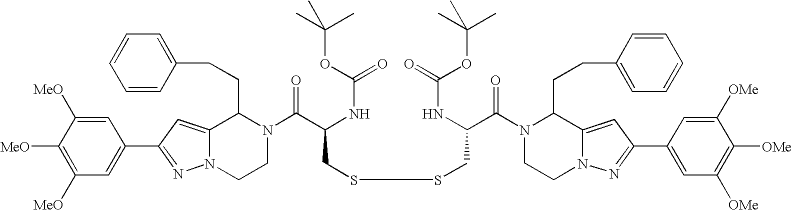

### 6a. (4RS)-4-(2-phenylethyl)-2-(3,4,5-trimethoxyphenyl)-4,5,6,7-tetrahydro pyrazolo[1,5-a]pyrazine

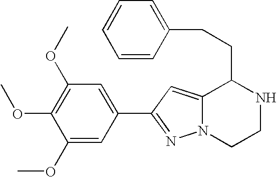

The compound below is synthesized according to a method analogous to that described in Example 1e.

Yellow oil—MH⁺ experimental=394.22; M theoretical=393.48

NMR-¹H (δ ppm, DMSO): 1.86-1.91 (m, 1H); 2.11-2.16 (m, 1H); 2.67 (br, 1H); 2.72-2.82 (m, 2H); 3.03 (ddd, 1H); 3.29-3.32 (m, 1H); 3.67 (br, 3H); 3.83 (br, 6H); 3.87 (dd, 1H); 3.96-4.03 (m, 2H); 6.57 (s, 1H); 7.04 (br, 2H); 7.16-7.20 (m, 1H); 7.26-7.31 (m, 4H).

NMR-¹³C (δ ppm, DMSO): 31.48; 36.61; 47.52; 51.95; 54.94; 55.82; 60.04; 98.02; 102.12; 125.77; 128.35; 128.40; 129.44; 136.77; 142.06; 143.35; 148.89; 153.05.

### 6b. tert-butyl {(1R)-1-[({(2R)-2-[(tert-butoxycarbonyl)amino]-3-oxo-3-[(4RS)-4-(2-phenylethyl)-2-(3,4,5-trimethoxyphenyl)-6,7-dihydropyrazolo[1,5-a]pyrazin-5(4H)-yl]propyl}dithio)methyl]-2-oxo-2-[(4RS)-4-(2-phenylethyl)-2-(3,4,5-trimethoxyphenyl)-6,7-dihydropyrazolo[1,5-a]pyrazin-5(4H)-yl]ethyl}carbamate

The compound of Example 6 is synthesized according to a method analogous to that described in Example 1f.

White solid—Melting point: 130-136° C.

[M+2H]²⁺ experimental=596.30; M theoretical=1191.47

NMR-¹H (δ ppm, DMSO): 1.28-1.37 (m, 18H); 2.07-2.11 (m, 4H); 2.63-2.69 (m, 4H); 2.61-3.21 (m, 4H); 3.66-3.84 (m, 18H); 3.99-4.83 (m, 10H); 5.71-5.74 (m, 2H); 6.65-6.67 (m, 2H); 7.00-7.25 (m, 4H); 7.14-7.25 (m, 10H); 7.55-7.57 (m, 2H).

NMR-¹³C (δ ppm, DMSO): 28.07; 31.63; 32.04; 36.11; 47.33; 48.63; 49.91; 51.45, 55.85; 60.00; 78.62; 99.56; 102.36; 125.75; 128.33; 128.92; 137.13; 140.28; 141.47; 149.76; 153.05; 155.40; 169.59.

### EXAMPLE 7

### tert-butyl {(1R)-1-[({(2R)-2-[(tert-butoxycarbonyl)amino]-3-oxo-3-[(4RS)-4-(2-phenylethyl)-2-(2-thienyl)-6,7-dihydropyrazolo[1,5-a]pyrazin-5(4H)-yl]propyl}dithio)methyl]-2-oxo-2-[(4RS)-4-(2-phenylethyl)-2-(2-thienyl)-6,7-dihydropyrazolo[1,5-a]pyrazin-5(4H)-yl]ethyl}carbamate

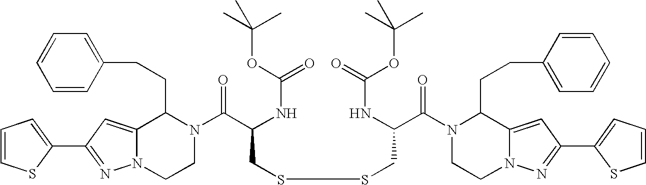

### 7a. (4RS)-4-(2-phenylethyl)-2-(2-thienyl)-4,5,6,7-tetrahydropyrazolo[1,5-a]pyrazine

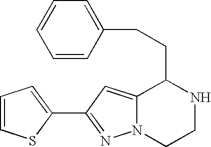

The compound below is synthesized according to a method analogous to that described in Example 1e.

Colourless oil—MH⁺ experimental=310.26; M theoretical=309.43

NMR-¹H (δ ppm, DMSO): 1.82-1.91 (m, 1H); 2.07-2.16 (m, 1H); 2.67 (br, 1H); 2.72-2.77 (m, 2H); 3.00 (ddd, 1H); 3.27-3.33 (m, 1H); 3.85 (d, 1H); 3.92-3.99 (m, 2H); 6.45 (s, 1H); 7.04 (dd, 1H); 7.17-7.20 (m, 1H); 7.25-7.29 (m, 4H); 7.32 (dd, 1H); 7.39 (dd, 1H).

NMR-¹³C (δ ppm, DMSO): 31.53; 36.56; 41.81; 47.60; 52.01; 97.87; 123.49; 124.57; 125.89; 127.70; 128.47; 137.19; 142.14; 143.59; 144.73.

### 7b. tert-butyl {(1R)-1-[({(2R)-2-[(tert-butoxycarbonyl)amino]-3-oxo-3-[(4RS)-4-(2-phenylethyl)-2-(2-thienyl)-6,7-dihydropyrazolo[1,5-a]pyrazin-5(4H)-yl]propyl}dithio)methyl]-2-oxo-2-[(4RS)-4-(2-phenylethyl)-2-(2-thienyl)-6,7-dihydro pyrazolo[1,5-a]pyrazin-5(4H)-yl]ethyl}carbamate

The compound of Example 7 is synthesized according to a method analogous to that described in Example 1f.

Light yellow solid—Melting point: 105-112° C.

[M+2H]²⁺ experimental=512.46; M theoretical=1023.38

NMR-¹H (δ ppm, DMSO): 1.26-1.42 (m, 18H); 2.13-2.15 (m, 4H); 2.67-2.73 (m, 4H); 2.91-3.24 (m, 4H); 3.88-4.85 (m, 10H); 5.76-5.78 (m, 2H); 6.60-6.62 (m, 2H); 7.11-7.14 (m, 2H); 7.21-7.32 (m, 10H); 7.42 (s, 2H); 7.50 (s, 2H) 7.65-7.67 (m, 2H).

### EXAMPLE 8

### tert-butyl {(1R)-1-[({(2R)-2-[(tert-butoxycarbonyl)amino]-3-[(4RS)-2-(2-furyl)-4-(2-phenylethyl)-6,7-dihydropyrazolo[1,5-a]pyrazin-5(4H)-yl]-3-oxopropyl}dithio)methyl]-2-[(4RS)-2-(2-furyl)-4-(2-phenylethyl)-6,7-dihydro pyrazolo[1,5-a]pyrazin-5(4H)-yl]-2-oxoethyl}carbamate

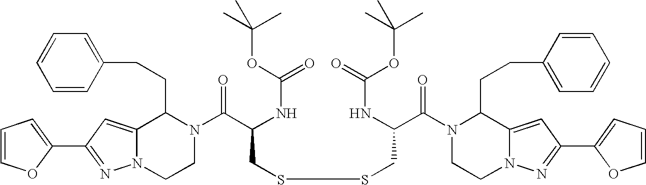

### 8a. (4RS)-2-(2-furyl)-4-(2-phenylethyl)-4,5,6,7-tetrahydropyrazolo[1,5-a]pyrazine

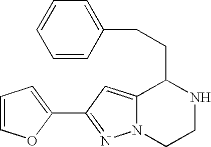

The compound below is synthesized according to a method analogous to that described in Example 1e.

Yellow oil—experimental=294.23; M theoretical=293.37

NMR-¹H (δ ppm, DMSO): 1.80-1.84 (m, 1H); 2.04-2.08 (m, 1H); 2.57 (br, 1H); 2.63-2.73 (m, 2H); 2.95 (ddd, 1H); 3.20-3.24 (m, 1H); 3.81 (dd, 1H); 3.88-3.94 (m, 2H); 6.28 (s, 1H); 6.45 (dd, 1H); 6.55 (dd, 1H); 7.09-7.24 (m, 5H); 7.57 (dd, 1H).

NMR-¹³C (δ ppm, DMSO): 31.29; 36.26; 41.59; 47.53; 51.79; 97.62; 104.97; 111.38; 125.68; 128.29; 128.81; 141.69; 141.92; 142.94; 149.13.

### 8b. tert-butyl {(1R)-1-[({(2R)-2-[(tert-butoxycarbonyl)amino]-3-[(4RS)-2-(2-furyl)-4-(2-phenylethyl)-6,7-dihydropyrazolo[1,5-a]pyrazin-5(4H)-yl]-3-oxopropyl}dithio)methyl]-2-[(4RS)-2-(2-furyl)-4-(2-phenylethyl)-6,7-dihydropyrazolo[1,5-a]pyrazin-5(4H)-yl]-2-oxoethyl}carbamate

The compound of Example 8 is synthesized according to a method analogous to that described in Example 1f.

Yellow oil—[M+2H]²⁺ experimental=496.37; M theoretical=991.24

NMR-¹H (δ ppm, DMSO): 1.29-1.36 (m, 18H); 2.03-2.19 (m, 4H); 2.61-2.73 (m, 4H); 2.89-3.21 (m, 4H); 3.79-4.81 (m, 10H); 5.51-5.71 (m, 2H); 6.40-6.47 (m, 2H); 6.50 (s, 2H); 6.63 (s, 2H); 7.11-7.28 (m, 10H); 7.51-7.58 (m, 2H); 7.62 (s, 2H).

NMR-¹³C (δ ppm, DMSO): 28.06; 31.38; 31.65; 38.20; 47.33; 48.37; 48.58; 49.95; 78.61; 99.50; 105, 36; 111.44; 125.75; 128.18; 141.39; 139.88; 142.19; 148.64; 155.39; 164.55.

### EXAMPLE 9

### tert-butyl {(1R)-1-[({(2R)-2-[(tert-butoxycarbonyl)amino]-3-oxo-3-[(4RS)-4-pentyl-2-phenyl-6,7-dihydropyrazolo[1,5-a]pyrazin-5(4H)-yl]propyl}dithio)methyl]-2-oxo-2-[(4RS)-4-pentyl-2-phenyl-6,7-dihydropyrazolo[1,5-a]pyrazin-5(4H)-yl]ethyl}carbamate

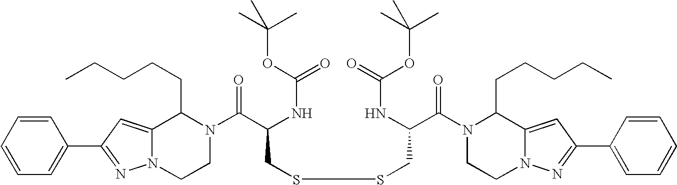

### 9a. (4RS)-4-pentyl-2-phenyl-4,5,6,7-tetrahydropyrazolo[1,5-a]pyrazine

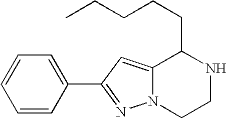

The compound below is synthesized according to a method analogous to that described in Example 1e.

Yellow oil—MH⁺ experimental=270.22; M theoretical=269.40

NMR-¹H (δ ppm, DMSO): 0.88 (t, 3H); 1.30-1.34 (m, 4H); 1.44-1.46 (m, 2H); 1.57-1.60 (m, 1H); 1.81-1.82 (m, 1H); 2.54 (br, 1H); 3.01 (ddd, 1H); 3.27 (dt, 1H); 3.85 (dd, 1H); 3.94-4.04 (m, 2H); 6.48 (s, 1H); 7.25 (tt, 1H); 7.36 (t, 2H); 7.75 (dd, 2H).

NMR-¹³C (δ ppm, DMSO): 13.91; 22.04; 24.89; 31.31; 34.56; 41.77; 47.52; 52.33; 97.63; 124.87; 127.11; 128.48; 133.69; 143.67; 148.76.

### 9b. tert-butyl {(1R)-1-[({(2R)-2-[(tert-butoxycarbonyl)amino]-3-oxo-3-[(4RS)-4-pentyl-2-phenyl-6,7-dihydropyrazolo[1,5-a]pyrazin-5(4H)-yl]propyl}dithio)methyl]-2-oxo-2-[(4RS)-4-pentyl-2-phenyl-6,7-dihydropyrazolo[1,5-a]pyrazin-5(4H)-yl]ethyl}carbamate

The compound of Example 9 is synthesized according to a method analogous to that described in Example 1f.

Yellow solid—Melting point: 94-100° C.

[M-1-211]²⁺ experimental=472.35; M theoretical=943.28

NMR-¹H (δ ppm, DMSO): 0.81-0.88 (m, 6H); 1.24-1.40 (m, 30H); 1.76 (m, 4H); 2.90-3.10 (m, 4H); 3.78-4.89 (m, 10H); 5.59-5.64 (m, 2H); 6.57-6.61 (m, 2H); 7.25-7.30 (m, 2H); 7.35-7.40 (m, 4H); 7.48-7.55 (m, 2H); 7.71-7.76 (m, 2H).

NMR-¹³C (δ ppm, DMSO): 13.80; 21.91; 27.30; 27.52; 28.06; 31.20; 33.78; 45.33, 47.20; 48.32; 78.52; 99.50; 124.84; 127.40; 128.56; 133.17; 140.65; 149.74; 155.29; 164.43.

### EXAMPLE 10

### tert-butyl {(1R)-1-[({(2R)-2-[(tert-butoxycarbonyl)amino]-3-[(4RS)-4-butyl-2-phenyl-6,7-dihydropyrazolo[1,5-a]pyrazin-5(4H)-yl]-3-oxopropyl}dithio)methyl]-2-[(4RS)-4-butyl-2-phenyl-6,7-dihydropyrazolo[1,5-a]pyrazin-5(4H)-yl]-2-oxoethyl}carbamate

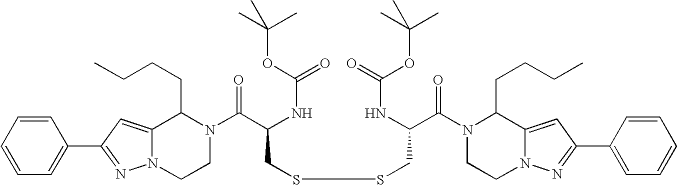

### 10a. (4RS)-4-butyl-2-phenyl-4,5,6,7-tetrahydropyrazolo[1,5-a]pyrazine

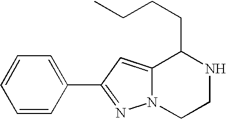

The compound below is synthesized according to a method analogous to that described in Example 1e.

Yellow oil—MH⁺ experimental=256.24; M theoretical=255.36

NMR-¹H (δ ppm, DMSO): 0.90 (t, 3H); 1.32-1.46 (m, 4H); 1.58-1.60 (m, 1H); 1.81-1.85 (m, 1H); 2.54 (br, 1H); 3.01 (ddd, 1H); 3.26 (dt, 1H); 3.85 (dd, 1H); 3.94-4.04 (m, 2H); 6.48 (s, 1H); 7.25 (tt, 1H); 7.36 (t, 2H); 7.75 (dd, 2H).

NMR-¹³C (δ ppm, DMSO): 13.93; 22.18; 27.45; 34.29; 41.77; 47.52; 52.30; 97.64; 124.87; 127.11; 128.48; 133.69; 143.67; 148.77.

### 10b. tert-butyl {(1R)-1-[({(2R)-2-[(tert-butoxycarbonyl)amino]-3-[(4RS)-4-butyl-2-phenyl-6,7-dihydropyrazolo[1,5-a]pyrazin-5(4H)-yl]-3-oxopropyl}dithio)methyl]-2-[(4RS)-4-butyl-2-phenyl-6,7-dihydropyrazolo[1,5-a]pyrazin-5(4H)-yl]-2-oxoethyl}carbamate

The compound of Example 10 is synthesized according to a method analogous to that described in Example 1f.

White solid—Melting point: 95-102° C.

[M+2H]²⁺ experimental=458.36; M theoretical=915.23

NMR-¹H (δ ppm, DMSO): 0.83-0.93 (m, 6H); 1.24-1.40 (m, 26H); 1.70 (m, 4H); 2.90-3.13 (m, 4H); 3.78-4.28 (m, 10H); 5.60-5.64 (m, 2H); 6.57-6.61 (m, 2H); 7.27-7.30 (m, 2H); 7.35-7.40 (m, 4H); 7.49-7.55 (m, 2H); 7.73-7.76 (m, 2H).

NMR-¹³C (δ ppm, DMSO): 13.78; 21.91; 27.52; 28.06; 31.20; 33.78; 45.22, 47.33; 48.31; 49.64; 78.71; 99.50; 124.84; 127.41; 128.56; 133.17; 140.58; 149.74; 155.14; 164.43.

### EXAMPLE 11

### tert-butyl {(1R)-1-[({(2R)-2-[(tert-butoxycarbonyl)amino]-3-[(4RS)-2-(2,4-dichlorophenyl)-4-(2-phenylethyl)-6,7-dihydropyrazolo[1,5-a]pyrazin-5(4H)-yl]-3-oxopropyl}dithio)methyl]-2-[(4RS)-2-(2,4-dichlorophenyl)-4-(2-phenylethyl)-6,7-dihydropyrazolo[1,5-a]pyrazin-5(4H)-yl]-2-oxoethyl}carbamate

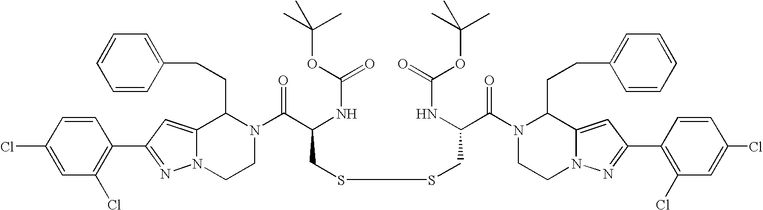

### 11a. (4RS)-2-(2,4-dichlorophenyl)-4-(2-phenylethyl)-4,5,6,7-tetrahydropyrazolo[1,5-a]pyrazine

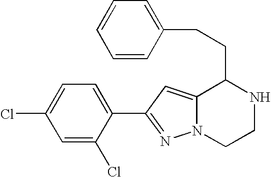

The compound below is synthesized according to a method analogous to that described in Example 1e.

MH⁺ experimental=372.13; M theoretical=372.30

### 11b. tert-butyl {(1R)-1-[({(2R)-2-[(tert-butoxycarbonyl)amino]-3-[(4RS)-2-(2,4-dichlorophenyl)-4-(2-phenylethyl)-6,7-dihydropyrazolo[1,5-a]pyrazin-5(4H)-yl]-3-oxopropyl}dithio)methyl]-2-[(4RS)-2-(2,4-dichlorophenyl)-4-(2-phenylethyl)-6,7-dihydropyrazolo[1,5-a]pyrazin-5(4H)-yl]-2-oxoethyl}carbamate

The compound of Example 11 is synthesized according to a method analogous to that described in Example 1f.

Yellow solid

[M+2H]²⁺ experimental=575.33; M theoretical=1149.10

### EXAMPLE 12

### (2R)-3-({(2R)-2-amino-3-[(4RS)-4-(cyclohexylmethyl)-2-phenyl-6,7-dihydropyrazolo[1,5-a]pyrazin-5(4H)-yl]-3-oxopropyl}dithio)-1-[(4RS)-4-(cyclohexylmethyl)-2-phenyl-6,7-dihydropyrazolo[1,5-a]pyrazin-5(4H)-yl]-1-oxopropan-2-amine hydrochloride

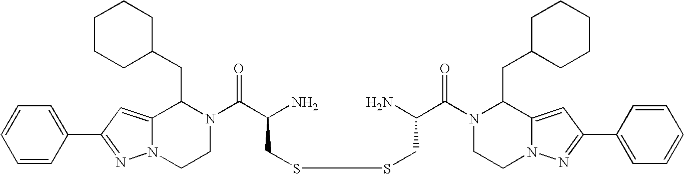

The compound of Example 1 (230 mg, 0.23 mmol) is dissolved in a mixture of ethyl acetate (5 mL) and ethanol (5 mL). HCl is added (2N in Et₂O, 2.9 mL, 5.8 mmol, 25 eq.) and the reaction medium is heated at 60° C. for 2 hours and 30 minutes (TLC eluent: DCM/MeOH=95/5, developer: ninhydrin), then the reaction medium is left to cool down to ambient temperature. The precipitate is collected by filtration, washed twice with Et₂O and dried under vacuum at 70° C. 129 mg (60%) of the compound of Example 12 is obtained.

Light yellow solid

Melting point: 218° C. (dec)

[M+2H]²⁺ experimental=398.40; M theoretical=795.13

NMR-¹H (δ ppm, DMSO): 0.88-1.85 (m, 26H); 3.19-3.58 (m, 4H); 4.22-4.99 (m, 10H); 5.01-5.19 (m, 2H); 5.57-5.68 (m, 2H); 6.55 (s, 2H); 7.26-7.30 (m, 2H); 7.36-7.38 (m, 4H); 7.73-7.77 (m, 4H).

### EXAMPLE 13

### (2R)-3-({(2R)-2-amino-3-oxo-3-[(4RS)-2-phenyl-4-(2-phenylethyl)-6,7-dihydropyrazolo[1,5-a]pyrazin-5(4H)-yl]propyl}dithio)-1-oxo-1-[(4RS)-2-phenyl-4-(2-phenylethyl)-6,7-dihydropyrazolo[1,5-a]pyrazin-5(4H)-yl]propan-2-amine hydrochloride

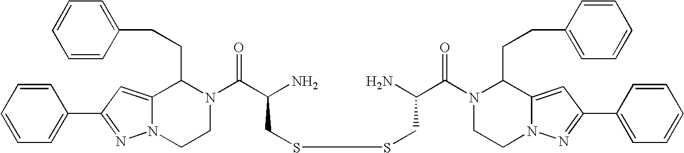

The above compound is synthesized according to a method analogous to that described in Example 12.

Yellow solid—Melting point: 208° C. (dec)

[M+2H]²⁺ experimental=406.41; M theoretical=811.09

NMR-¹H (δ ppm, DMSO): 2.08-2.14 (m, 4H); 2.66-2.75 (m, 4H); 3.25-3.42 (m, 4H); 4.23-4.86 (m, 10H); 5.60-5.63 (m, 2H); 6.67 (s, 2H); 7.14-7.30 (m, 12H); 7.34-7.40 (m, 4H); 7.73-7.81 (m, 4H); 8.60-8.80 (m, 6H).

NMR-¹³C (δ ppm, DMSO): 32.41; 36.73; 48.16; 49.68; 50.38; 125.93; 126.71; 128.46; 129.16; 129.52; 134.02; 140.47; 142.24; 150.77; 167.15.

### EXAMPLE 14

### (2R)-3-({(2R)-2-amino-3-oxo-3-[(4RS)-4-(2-phenylethyl)-2-pyridin-4-yl-6,7-dihydropyrazolo[1,5-a]pyrazin-5(4H)-yl]propyl}dithio)-1-oxo-1-[(4RS)-4-(2-phenylethyl)-2-pyridin-4-yl-6,7-dihydropyrazolo[1,5-a]pyrazin-5(4H)-yl]propan-2-amine hydrochloride

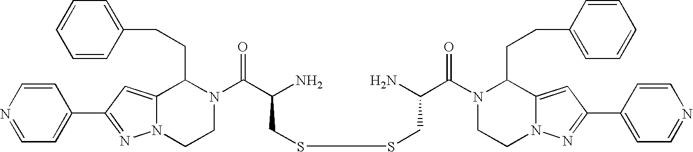

The above compound is synthesized according to a method analogous to that described in Example 12.

White solid—Melting point: 227° C. (dec)

[M+2H]²⁺ experimental=407.40; M theoretical=813.07

NMR-¹H (δ ppm, DMSO): 2.12 (m, 4H); 2.73-2.77 (m, 4H); 3.26-5.82 (m, 14H); 5.61-5.89 (m, 2H); 6.67 (s, 2H); 7.18-7.42 (m, 12H); 8.24-8.32 (m, 4H); 8.78-8.88 (m, 10H).

NMR-¹³C (δ ppm, DMSO): 34.11; 36.07; 44.91; 48.48; 49.04; 52.06; 104.48; 122.06; 126.57; 126.72; 139.80; 141.67; 142.79; 145.56; 148.01; 166.88.

### EXAMPLE 15

### (2R)-3-({(2R)-2-amino-3-oxo-3-[(4RS)-4-(2-phenylethyl)-2-pyridin-3-yl-6,7-dihydropyrazolo[1,5-a]pyrazin-5(4H)-yl]propyl}dithio)-1-oxo-1-[(4RS)-4-(2-phenylethyl)-2-pyridin-3-yl-6,7-dihydropyrazolo[1,5-a]pyrazin-5(4H)-yl]propan-2-amine hydrochloride

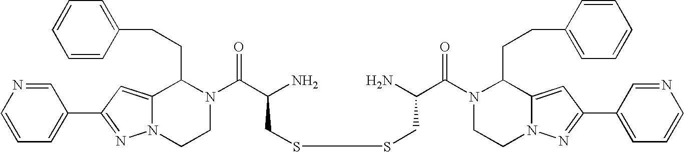

The above compound is synthesized according to a method analogous to that described in Example 12.

Light yellow solid—Melting point: 223° C. (dec)

[M+2H]²⁺ experimental=407.44; M theoretical=813.07

NMR-¹H (δ ppm, DMSO): 2.09-2.12 (m, 4H); 2.71-2.77 (m, 4H); 3.20-3.57 (m, 4H); 3.58-5.09 (m, 10H); 5.60-5.68 (m, 2H); 6.99-7.07 (s, 2H); 7.17-7.29 (m, 10H); 7.97-7.99 (m, 2H); 8.73-8.90 (m, 10H); 9.21-9.24 (m, 2H).

### EXAMPLE 16

### (2R)-3-({(2R)-2-amino-3-[(4RS)-2-(1,3-benzodioxol-5-yl)-4-(2-phenylethyl)-6,7-dihydropyrazolo[1,5-a]pyrazin-5(4H)-yl]-3-oxopropyl}dithio)-1-[(4RS)-2-(1,3-benzodioxol-5-yl)-4-(2-phenylethyl)-6,7-dihydropyrazolo[1,5-a]pyrazin-5(4H)-yl]-1-oxopropan-2-amine hydrochloride

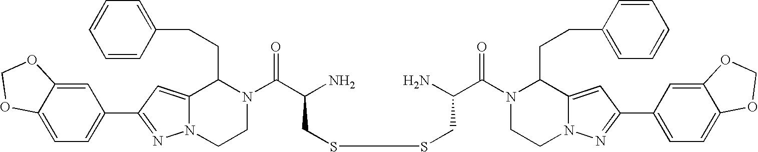

The above compound is synthesized according to a method analogous to that described in Example 12.

Beige solid—Melting point: 221° C. (dec)

[M+2H]²⁺ experimental=450.17; M theoretical=899.11

NMR-¹H (δ ppm, DMSO): 2.08-2.11 (m, 4H); 2.65-2.77 (m, 4H); 3.21-3.52 (m, 4H); 3.89-4.83 (m, 10H); 5.50-5.74 (m, 2H); 6.53-6.64 (s, 2H); 6.88-6.93 (m, 2H); 7.16-7.33 (m, 14H); 8.66-8.86 (m, 6H).

NMR-¹³C (δ ppm, DMSO): 31.60; 35.78; 47.18; 48.54; 48.67; 49.45; 99.27; 100.97; 105.39; 108.42; 118.69; 125.87; 127.45; 128.23; 139.55; 141.36; 146.74; 147.58; 149.69; 166.29.

### EXAMPLE 17

### (2R)-3-({(2R)-2-amino-3-oxo-3-[(4RS)-4-(2-phenylethyl)-2-(3,4,5-trimethoxyphenyl)-6,7-dihydropyrazolo[1,5-a]pyrazin-5(4H)-yl]propyl}dithio)-1-oxo-1-[(4RS)-4-(2-phenylethyl)-2-(3,4,5-trimethoxyphenyl)-6,7-dihydropyrazolo[1,5-a]pyrazin-5(4H)-yl]propan-2-amine hydrochloride

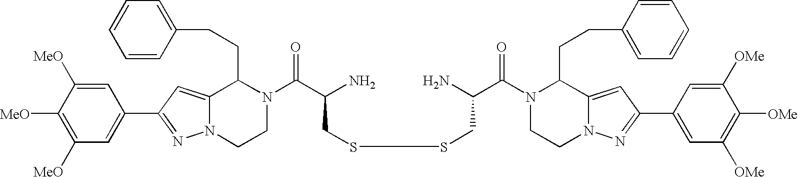

The above compound is synthesized according to a method analogous to that described in Example 12.

Light yellow solid—Melting point: 211° C. (dec)

[M+2H]²⁺ experimental=496.38; M theoretical=991.25

NMR-¹H (δ ppm, DMSO): 2.1 (m, 4H); 2.65-2.74 (m, 4H); 3.20-3.41 (m, 4H); 3.70-3.71 (m, 6H); 3.75-3.80 (m, 12H); 3.93-5.78 (m, 12H); 6.64-6.75 (m, 2H); 6.96-7.03 (m, 4H); 7.15-7.25 (m, 10H); 8.71-8.90 (m, 6H).

NMR-¹³C (δ ppm, DMSO): 31.59; 32.04; 35.99; 47.28; 48.63; 48.87; 49.53; 55.86; 60.05; 99.80; 102.42; 125.80; 128.25; 128.80; 137.16; 139.62; 141.43; 149.78; 153.06; 166.99.

### EXAMPLE 18

### (2R)-3-({(2R)-2-amino-3-oxo-3-[(4RS)-4-(2-phenylethyl)-2-(2-thienyl)-6,7-dihydropyrazolo[1,5-a]pyrazin-5(4H)-yl]propyl}dithio)-1-oxo-1-[(4RS)-4-(2-phenylethyl)-2-(2-thienyl)-6,7-dihydropyrazolo[1,5-a]pyrazin-5(4H)-yl]propan-2-amine hydrochloride

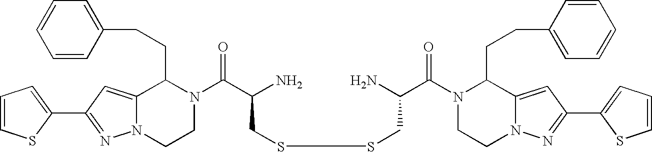

The above compound is synthesized according to a method analogous to that described in Example 12.

Light yellow solid—Melting point: 218° C. (dec)

[M+2H]²⁺ experimental=412.36; M theoretical=823.14

NMR-¹H (δ ppm, DMSO): 2.04-2.07 (m, 4H); 2.59-2.69 (m, 4H); 3.18-3.49 (m, 4H); 3.81-4.89 (m, 10H); 5.51-5.73 (m, 2H); 6.51-6.52 (m, 2H); 7.00-7.02 (m, 2H); 7.11-7.23 (m, 10H); 7.30-7.33 (m, 2H); 7.37-7.39 (m, 2H); 8.61-8.65 (m, 6H).

### EXAMPLE 19

### (2R)-3-({(2R)-2-amino-3-[(4RS)-2-(2-furyl)-4-(2-phenylethyl)-6,7-dihydropyrazolo[1,5-a]pyrazin-5(4H)-yl]-3-oxopropyl}dithio)-1-[(4RS)-2-(2-furyl)-4-(2-phenylethyl)-6,7-dihydropyrazolo[1,5-a]pyrazin-5(4H)-yl]-1-oxopropan-2-amine hydrochloride

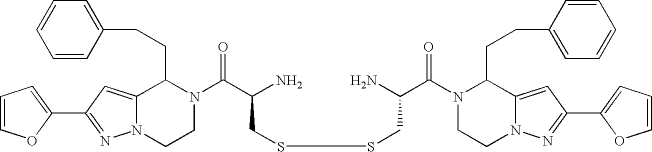

The above compound is synthesized according to a method analogous to that described in Example 12.

Light yellow solid—Melting point: 207° C. (dec)

[M+2H]²⁺ experimental=396.37; M theoretical=791.01

NMR-¹H (δ ppm, DMSO): 1.96, 2, 21 (m, 4H); 2.62-2.71 (m, 4H); 3.23-3.58 (m, 4H); 3.92-4.93 (m, 10H); 5.61-5.76 (m, 2H); 6.40-6.53 (m, 4H); 6.67 (m, 2H); 7.11-7.28 (m, 10H); 7.67 (m, 2H); 8.69-8.92 (m, 6H).

NMR-¹³C (δ ppm, DMSO): 31.43; 31.87; 35.72; 47.30; 48.48; 48.63; 49.41; 99.44; 111.52; 125.79; 128.22; 139.36; 141.35; 142.29; 148.45; 166.29.

### EXAMPLE 20

### (2R)-3-({(2R)-2-amino-3-oxo-3-[(4RS)-4-pentyl-2-phenyl-6,7-dihydropyrazolo[1,5-a]pyrazin-5(4H)-yl]propyl}dithio)-1-oxo-1-[(4RS)-4-pentyl-2-phenyl-6,7-dihydropyrazolo[1,5-a]pyrazin-5(4H)-yl]propan-2-amine hydrochloride

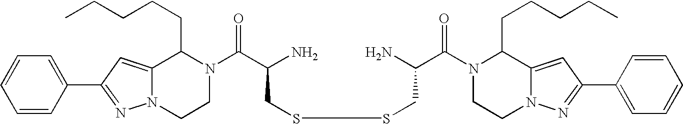

The above compound is synthesized according to a method analogous to that described in Example 12.

Beige solid—Melting point: 199-204° C. (dec)

[M+2H]²⁺ experimental=372.30; M theoretical=743.06

NMR-¹H (δ ppm, DMSO): 0.78-0.87 (m, 6H); 1.25-1.38 (m, 12H); 1.75-1.83 (m, 4H); 3.19-3.43 (m, 4H); 3.84-4.39 (m, 10H); 5.44-5.67 (m, 2H); 6.56-6.65 (m, 2H); 7.26-7.30 (m, 2H); 7.34-7.39 (m, 4H); 7.73-7.77 (m, 4H); 8.63-8.80 (m, 6H).

NMR-¹³C (δ ppm, DMSO): 13.93; 21.86; 24.88; 30.86; 31.09; 33.84; 47.17; 48.50; 48.67; 99.51; 124.87; 127.52; 128.48; 133.10; 139.83; 148.76; 165.99.

### EXAMPLE 21

### (2R)-3-({(2R)-2-amino-3-[(4RS)-4-butyl-2-phenyl-6,7-dihydro pyrazolo[1,5-a]pyrazin-5(4H)-yl]-3-oxopropyl}dithio)-1-[(4RS)-4-butyl-2-phenyl-6,7-dihydropyrazolo[1,5-a]pyrazin-5(4H)-yl]-1-oxopropan-2-amine hydrochloride

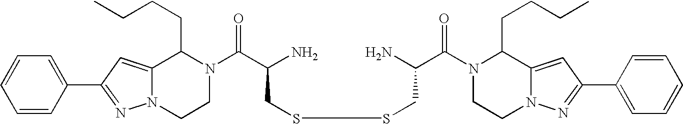

The above compound is synthesized according to a method analogous to that described in Example 12.

Beige solid—Melting point: 210-215° C. (dec)

[M+2H]²⁺ experimental=358.28; M theoretical=715.00

NMR-¹H (δ ppm, DMSO): 0.82-0.91 (m, 6H); 1.25-1.36 (m, 8H); 1.75-1.84 (m, 4H); 3.21-3.44 (m, 4H); 3.84-4.95 (m, 10H); 5.45-5.55 (m, 2H); 6.60-6.65 (m, 2H); 7.26-7.29 (m, 2H); 7.34-7.39 (m, 4H); 7.73-7.77 (m, 4H); 8.63-8.81 (m, 6H).

NMR-¹³C (δ ppm, DMSO): 13.83; 21.97; 27.33; 27.39; 33.62; 47.15; 48.75; 49.48; 99.51; 124.99; 127.52; 128.59; 133.10; 139.78; 149.78; 165.98.

### EXAMPLE 22

### (2R)-3-({(2R)-2-amino-3-[(4RS)-2-(2,4-dichlorophenyl)-4-(2-phenylethyl)-6,7-dihydropyrazolo[1,5-a]pyrazin-5(4H)-yl]-3-oxopropyl}dithio)-1-[(4RS)-2-(2,4-dichlorophenyl)-4-(2-phenylethyl)-6,7-dihydropyrazolo[1,5-a]pyrazin-5(4H)-yl]-1-oxopropan-2-amine hydrochloride

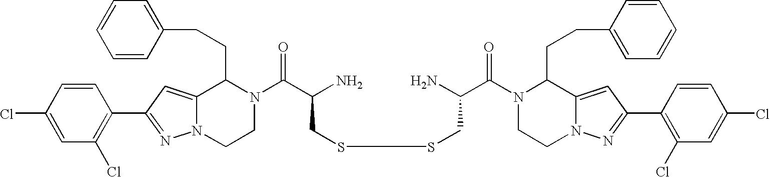

The above compound is synthesized according to a method analogous to that described in Example 12.

Pale yellow solid—Melting point: 198-202° C.

MH⁺ experimental=947.28; M theoretical=948.87

NMR-¹H (δ ppm, DMSO): 2.09-2.15 (m, 4H); 2.66-2.76 (m, 4H); 3.27-3.42 (m, 4H); 3.98-4.42 (m, 10H); 6.66-6.73 (m, 2H); 7.16-7.28 (m, 10H); 7.43-7.45 (m, 2H); 7.66-7.69 (m, 2H); 7.76-7.79 (m, 2H); 8.60-8.79 (m, 6H).

NMR-¹³C (δ ppm, DMSO): 32.41; 36.56; 48.23; 49.72; 50.27; 104.29; 126.74; 128.41; 129.17; 130.55; 131.68; 132.28; 132.63; 133.67; 139.93; 142.18; 147.29; 167.21.

### EXAMPLE 23

### tert-butyl {(1R)-1-[({(2R)-2-[(tert-butoxycarbonyl)amino]-3-[(4S)-4-(cyclohexylmethyl)-2-phenyl-6,7-dihydropyrazolo[1,5-a]pyrazin-5(4H)-yl]-3-oxopropyl}dithio)methyl]-2-[(4S)-4-(cyclohexylmethyl)-2-phenyl-6,7-dihydropyrazolo[1,5-a]pyrazin-5(4H)-yl]-2-oxoethyl}carbamate

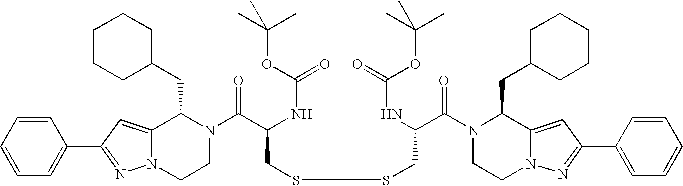

### 23a. (4S)-4-(cyclohexylmethyl)-2-phenyl-4,5,6,7-tetrahydropyrazolo[1,5-a]pyrazine

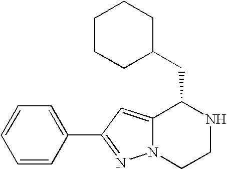

The compound of Example 1c (823 mg, 2.0 mmol) in solution in formic acid (7.4 mL; 196 mmol; 9 vol) is reacted under an inert atmosphere at ambient temperature, over 27 hours (TLC, eluent: DCM/MeOH=98/2). The reaction medium is cooled down to 0° C. and triethylamine (10.9 mL; 75 mmol; 3.6 vol) is added dropwise then the temperature is left to return to ambient. The hydrogen transfer catalyst is prepared by stirring bis((η⁶-p-cymene)dichlororuthenium) (3 mg; 5 μmol; 0.25% eq), (1R,2R)-TsDPEN (N-(4-toluenesulphonyl)-1,2-diphenylethylenediamine, 3.7 mg; 10 μmol; 0.50% eq) and a drop of Et₃N in anhydrous acetonitrile (4 mL) under argon for 40 minutes at 28° C. The catalyst solution is added to the reaction medium which is stirred at 28° C. for 20 hours (TLC, eluent: DCM/MeOH=95/5). The reaction medium (pH 9-10) is basified by adding a saturated solution of sodium carbonate followed by extraction three times with DCM. The organic phase is dried over sodium sulphate, then filtered and concentrated under vacuum. The residue is purified on SiO₂ (eluent: DCM/MeOH=98/2) in order to obtain the compound of Example 23a (207 mg, 35%) and its formamide derivative (422 mg, 65%). The formamide derivative can be hydrolyzed to the compound of Example 23a by heating under reflux of ethanol in the presence of 10% hydrochloric acid without significant loss of enantiomeric purity.

A white solid; Melting point: 113° C.

MH⁺ experimental=296.25; M theoretical=295.43

NMR-¹H (δ ppm, DMSO): 0.91-1.85 (m, 13H); 2.44 (s, 1H); 3.01 (ddd, 1H); 3.27 (dt, 1H); 3.91-4.00 (m, 3H); 6.45 (s, 1H); 7.25 (t, 1H); 7.36 (t, 2H); 7.75 (d, 2H).

NMR-¹³C (δ ppm, DMSO): 25.30; 25.56; 32.22; 33.54; 34.32; 42.05; 47.47; 49.66; 97.50; 124.86; 127.10; 128.48; 133.69; 144.17; 149.12.

### 23b. tert-butyl {(1R)-1-[({(2R)-2-[(tert-butoxycarbonyl)amino]-3-[(4S)-4-(cyclohexylmethyl)-2-phenyl-6,7-dihydropyrazolo[1,5-a]pyrazin-5(4H)-yl]-3-oxopropyl}dithio)methyl]-2-[(4S)-4-(cyclohexylmethyl)-2-phenyl-6,7-dihydro pyrazolo[1,5-a]pyrazin-5(4H)-yl]-2-oxoethyl}carbamate

A procedure analogous to the preparation of compound 1f is followed.

A white solid; Melting point: 106-108° C.

[M+2H]²⁺ experimental=498.35; M theoretical=995.36

NMR-¹H (δ ppm, DMSO): 1.24-1.40 (m, 18H); 0.85-1.93 (m, 26H); 2.79-3.07 (m, 4H); 3.41-4.77 (m, 10H); 5.73-5.75 (m, 2H); 6.52-6.55 (m, 2H) 7.24-7.28 (m, 2H) 7.45-7.48 (m, 2H); 7.71-7.77 (m, 4H).

NMR-¹³C (δ ppm, DMSO): 25.40; 26.05; 27.98; 32.51; 33.07; 33.16; 41.68; 46.09; 47.34; 49.65; 97.50; 124.86; 127.10; 128.48; 133.69; 144.17; 149.12.

### EXAMPLE 24

### (2R)-3-({(2R)-2-amino-3-[(4S)-4-(cyclohexylmethyl)-2-phenyl-6,7-dihydropyrazolo[1,5-a]pyrazin-5(4H)-yl]-3-oxopropyl}dithio)-1-[(4S)-4-(cyclohexylmethyl)-2-phenyl-6,7-dihydropyrazolo[1,5-a]pyrazin-5(4H)-yl]-1-oxopropan-2-amine hydrochloride

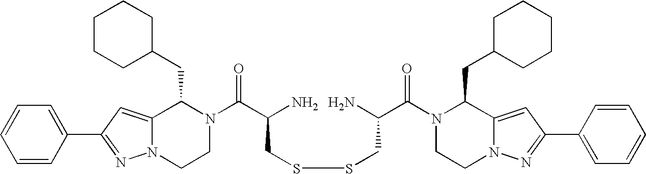

A procedure analogous to the preparation of compound 12 is followed.

Pale yellow solid

Melting point: 214-219° C.

[M+2H]²⁺ experimental=398.40; M theoretical=795.13

NMR-¹H (δ ppm, DMSO): 0.88-1.90 (m, 26H); 3.18-3.42 (m, 4H); 3.87-4.80 (m, 10H); 5.22-5.63 (m, 2H); 6.43-6.58 (m, 2H); 7.26-7.30 (m, 2H); 7.34-7.39 (m, 4H); 7.73-7.77 (m, 4H); 8.70-8.75 (m, 6H).

NMR-¹³C (δ ppm, DMSO): 25.35; 26.09; 32.52; 32.86; 38.08; 41.37; 46.98; 47.19; 48.62; 99.41; 124.85; 127.53; 128.22; 133.08; 139.94; 149.83; 165.86.

### EXAMPLE 25

### tert-butyl {(1R)-1-[({(2R)-2-[(tert-butoxycarbonyl)amino]-3-[(4R)-4-(cyclohexylmethyl)-2-phenyl-6,7-dihydropyrazolo[1,5-a]pyrazin-5(4H)-yl]-3-oxopropyl}dithio)methyl]-2-[(4R)-4-(cyclohexylmethyl)-2-phenyl-6,7-dihydropyrazolo[1,5-a]pyrazin-5(4H)-yl]-2-oxoethyl}carbamate

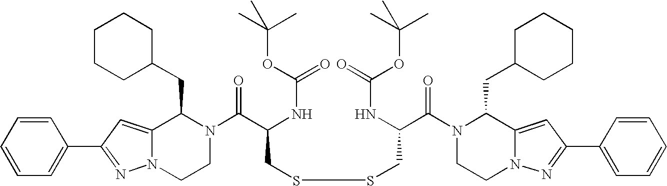

### 25a. (4R)-4-(cyclohexylmethyl)-2-phenyl-4,5,6,7-tetrahydropyrazolo[1,5-a]pyrazine

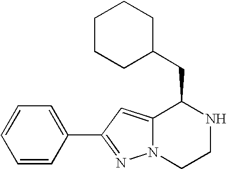

A procedure analogous to the preparation of Example 23a is followed, using (1S,2S)-TsDPEN instead of (1R,2R)-TsDPEN. A white solid is obtained; Melting point: 117-118° C.

MH⁺ experimental=296.27; M theoretical=295.43

NMR-¹H (δ ppm, DMSO): 0.88-1.88 (m, 13H); 2.45 (s, 1H); 3.03 (ddd, 1H); 3.26 (dt, 1H); 3.91-4.00 (m, 3H); 6.45 (s, 1H); 7.25 (t, 1H); 7.37 (t, 2H); 7.75 (d, 2H).

NMR-¹³C (δ ppm, DMSO): 25.60; 25.89; 26.16; 31.83; 33.15; 33.93; 41.65; 42.38; 47.47; 49.67; 97.50; 124.86; 127.10; 128.48; 133.69; 144.17; 148.72.

### 25b. tert-butyl {(1R)-1-[({(2R)-2-[(tert-butoxycarbonyl)amino]-3-[(4R)-4-(cyclohexylmethyl)-2-phenyl-6,7-dihydropyrazolo[1,5-a]pyrazin-5(4H)-yl]-3-oxopropyl}dithio)methyl]-2-[(4R)-4-(cyclohexylmethyl)-2-phenyl-6,7-dihydro pyrazolo[1,5-a]pyrazin-5(4H)-yl]-2-oxoethyl}carbamate

A procedure analogous to the preparation of compound 1f is followed.

A white solid; Melting point: 114-139° C.

[M+2H]²⁺ experimental=498.37; M theoretical=995.36

NMR-¹H (δ ppm, DMSO): 1.24-1.40 (m, 18H); 0.85-1.93 (m, 26H); 2.79-3.07 (m, 4H); 3.41-4.77 (m, 10H); 5.81-5.83 (m, 2H); 6.61-6.64 (m, 2H) 7.33-7.36 (m, 2H) 7.41-7.46 (m, 2H); 7.79-7.83 (m, 4H).

### EXAMPLE 26

### (1R)-3-({(2R)-2-amino-3-[(4R)-4-(cyclohexylmethyl)-2-phenyl-6,7-dihydropyrazolo[1,5-a]pyrazin-5(4H)-yl]-3-oxopropyl}dithio)-1-[(4R)-4-(cyclohexylmethyl)-2-phenyl-6,7-dihydropyrazolo[1,5-a]pyrazin-5(4H)-yl]-1-oxopropan-2-amine hydrochloride

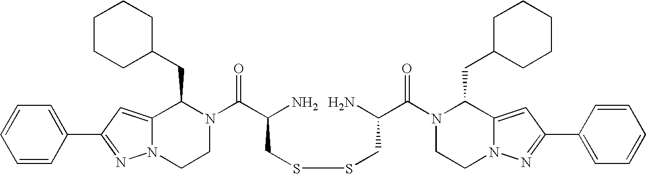

A procedure analogous to the preparation of Example 12 is followed.

Yellow solid white—Melting point: 212-217° C.

[M+2H]²⁺ experimental=398.41; M theoretical=795.13

NMR-¹H (δ ppm, DMSO): 0.91-1.97 (m, 26H); 3.33-3.57 (m, 4H); 3.86-4.98 (m, 10H); 5.83 (m, 2H); 6.54-6.61 (m, 2H); 7.20-7.40 (m, 6H); 7.73-7.77 (m, 4H); 8.70-8.75 (m, 6H).

NMR-¹³C (δ ppm, DMSO): 25.55; 25.78; 26.04; 32.16; 33.10; 33.34; 38.00; 41.67; 46.37; 47.36; 48.37; 99.29; 125.02; 127.52; 128.60; 133.13; 140.40; 149.79; 166.78.

### EXAMPLE 27

### tert-butyl {(1R)-1-[({(2R)-2-[(tert-butoxycarbonyl)amino]-3-[(4RS)-4-(cyclohexylmethyl)-2-phenyl-7,8-dihydro-4H-pyrazolo[1,5-a][1,4]diazepin-5(6H)-yl]-3-oxopropyl}dithio)methyl]-2-[(4RS)-4-(cyclohexylmethyl)-2-phenyl-7,8-dihydro-4H-pyrazolo[1,5-a][1,4]diazepin-5(6H)-yl]-2-oxoethyl}carbamate

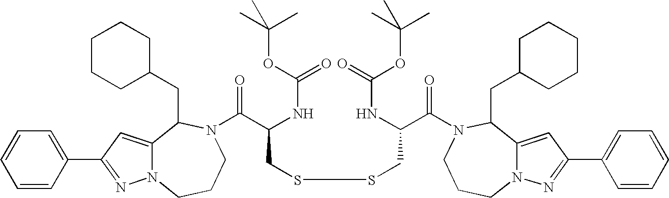

### 27a. tert-butyl [3-(5-{[methoxy(methyl)amino]carbonyl}-3-phenyl-1H-1-pyrazol-1-yl)propyl]carbamate

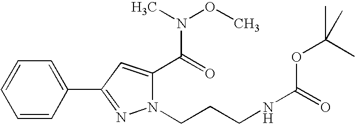

The compound of Example 1a (2.65 g, 11.5 mmol) in DMF (50 mL) is reacted with tert-butyl (3-bromopropyl)carbamate (3.55 g, 14.9 mmol, 1.3 eq.) in the presence of potassium carbonate (1.90 g, 13.7 mmol, 1.2 eq.). The reaction medium is heated at 110° C. for 6 hours (TLC, eluent: heptane/AcOEt=98/2). The DMF is then evaporated off and the residue is dissolved in AcOEt, followed by washing twice with water. The organic phase is dried over sodium sulphate, then filtered and concentrated under vacuum. The residue is purified on SiO₂ (eluent: heptane/AcOEt=60/40) in order to obtain the compound of Example 27a (3.93 g, 72%) in the form of a translucent oil.

MH⁺ experimental=389.28; M theoretical=388.46

NMR-¹H (δ ppm, DMSO): 1.36 (s, 9H); 1.87-1.94 (tt, 2H); 2.91-2.97 (m, 2H); 3.30 (s, 3H); 3.68 (s, 3H); 4.35 (t, 2H); 6.81 (br, 1H); 7.13 (s, 1H); 7.31 (m, 1H); 7.41 (m, 2H); 7.83 (m, 2H).

NMR-¹³C (δ ppm, DMSO): 28.38; 30.74; 37.60; 49.04; 61.52; 77.68; 105.36; 125.35; 127.96; 128.83; 132.71; 134.88; 148.89; 155.71; 159.98.

### 27b. tert-butyl {3-[5-(cyclohexylacetyl)-3-phenyl-1H-pyrazol-1-yl]propyl}carbamate

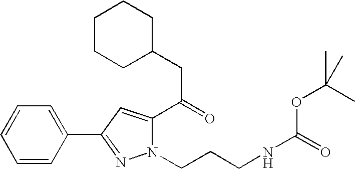

A procedure analogous to the preparation of Example 1c is followed. A yellow oil is obtained.

NMR-¹H (δ ppm, DMSO): 0.85-1.89 (m, 11H); 1.37 (s, 9H); 2.80 (d, 2H); 2.95 (m, 2H); 4.47 (t, 2H); 6.80 (br, 1H); 7.33 (m, 1H); 7.43 (m 2H); 7.66 (s, 1H); 7.85 (m, 2H).

NMR-¹³C (δ ppm, DMSO): 25.57; 25.76; 27.44; 28.19; 30.41; 32.50; 34.17; 37.37; 47.41; 49.52; 77.48; 109.28; 125.12; 127.92; 128.68; 132.20; 139, 77; 148.69; 155.49; 191.41.

MH⁺ experimental=426.31; M theoretical=425.57

### 27c. 4-(cyclohexylmethyl)-2-phenyl-7,8-dihydro-6H-pyrazolo[1,5-a][1,4]diazepine

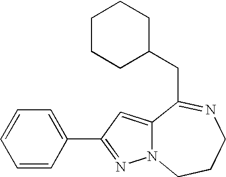

A procedure analogous to the preparation of Example 1d is followed. A yellow oil is obtained.

MH⁺ experimental=308.27; M theoretical=307.44

NMR-¹H (δ ppm, DMSO): 0.87-1.65 (m, 10H); 1.74-1.81 (m, 1H); 2.12-2.15 (m, 2H); 2.73-2.78 (m, 2H); 3.53-3.56 (m, 2H); 4.26 (t, 2H); 7.02 (s, 1H); 7.25 (m, 1H); 7.35 (m, 2H); 7.80 (m, 2H).

### 27d. (4RS)-4-(cyclohexylmethyl)-2-phenyl-5,6,7,8-tetrahydro-4H-pyrazolo[1,5-a][1,4]diazepine

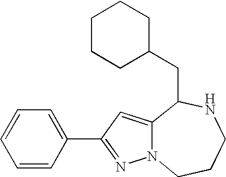

A procedure analogous to the preparation of Example 1e is followed. A translucent oil is obtained.

MH⁺ experimental=310.31; M theoretical=309.45

NMR-¹H (δ ppm, DMSO): 0.89-1.85 (m, 15H); 2.49 (br, 1H); 2.88-3.29 (ddd, 2H); 3.71-4.39 (m, 3H); 6.46 (s, 1H); 7.25 (t, 1H); 7.36 (t, 2H); 7.75 (d, 2H).

NMR-¹³C (δ ppm, DMSO): 25.73; 25.90; 26.18; 29.83; 32.13; 33.52; 33.68; 41.02; 49.86; 50.60; 51.98; 99.89; 124.77; 126.92; 128.46; 133.72; 147.07; 150.29.

### 27e. tert-butyl {(1R)-1-[({(2R)-2-[(tert-butoxycarbonyl)amino]-3-[(4RS)-4-(cyclohexyl methyl)-2-phenyl-7,8-dihydro-4H-pyrazolo[1,5-a][1,4]diazepin-5(6H)-yl]-3-oxopropyl}dithio)methyl]-2-[(4RS)-4-(cyclohexylmethyl)-2-phenyl-7,8-dihydro-4H-pyrazolo[1,5-a][1,4]diazepin-5(6H)-yl]-2-oxoethyl}carbamate

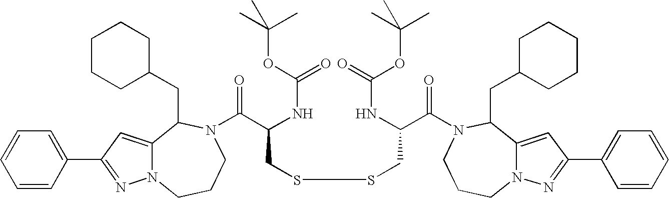

A procedure analogous to the preparation of Example 1f is followed. A pale yellow solid is obtained.

Melting point: 105-116° C.

[M+2H]²⁺ experimental=512.42; M theoretical=1023.41

NMR-¹H (δ ppm, DMSO): 1.31-1.41 (m, 18H); 0.70-1.96 (m, 30H); 2.7-3.3 (m, 4H); 3.5-4.5 (m, 10H); 5.74 (m, 2H); 6.6 (m, 2H) 7.23-7.27 (m, 2H) 7.33-7.39 (m, 4H); 7.72-7.75 (m, 4H).

NMR-¹³C (δ ppm, DMSO): 25.49; 35.74; 26.01; 27.97; 28.08; 28.59; 31.71; 32.92; 33.06; 33.41; 38.20; 50.07; 51.13; 54.85; 78.54; 99.49; 124.80; 124.89; 127.20; 128.52; 133.14; 133.18; 144.01; 147.71; 154.88; 155.03.

### EXAMPLE 28

### {(1R)-1-[({(2R)-2-amino-3-[(4RS)-4-(cyclohexylmethyl)-2-phenyl-7,8-dihydro-4H-pyrazolo[1,5-a][1,4]diazepin-5(6H)-yl]-3-oxopropyl}dithio)methyl]-2-[(4RS)-4-(cyclohexylmethyl)-2-phenyl-7,8-dihydro-4H-pyrazolo[1,5-a][1,4]diazepin-5(6H)-yl]-2-oxoethyl}amine hydrochloride

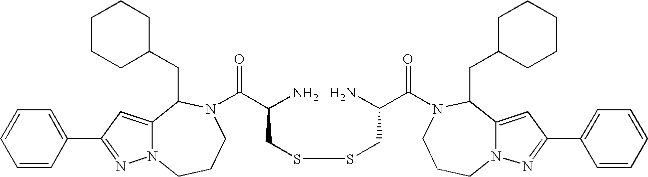

A procedure analogous to the preparation of Example 12 is followed.

Pale pink solid—Melting point: 198-208° C.

[M+2H]²⁺ experimental=412.37; M theoretical=823.18

NMR-¹H (δ ppm, DMSO): 0.89-2.2 (m, 30H); 3.18-3.42 (m, 4H); 3.87-4.80 (m, 10H); 5.9 (m, 2H); 6.63-6.68 (m, 2H); 7.23-7.27 (m, 2H); 7.34-7.38 (m, 4H); 7.71-7.76 (m, 4H); 8.56-8.74 (m, 6H).

NMR-¹³C (δ ppm, DMSO): 25.41; 25.62; 25.73; 26.04; 31.55; 32.12; 32.92; 33.13; 33.20; 37.93; 48.86; 51.16; 99.49; 124.82; 127.32; 128.65; 133.09; 143.56; 147.80; 166.99.

### EXAMPLE 29

### (2R)-2-amino-3-[(4RS)-2-(2-furyl)-4-(2-phenylethyl)-6,7-dihydropyrazolo[1,5-a]pyrazin-5(4H)-yl]-3-oxopropane-1-thiol hydrochloride

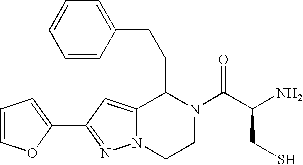

The compound of Example 19 (93.7 mg; 0.100 mmol) is dissolved in ethanol, dithiothreitol (23 mg; 0.150 mmol) is added and the reaction medium is heated under reflux for 5 hours under an argon atmosphere, then at ambient temperature for 12 hours. The reaction is cooled down to 5° C. then TBME and then Et₂O are added. The precipitate is collected on frit and washed with Et₂O, then dried under vacuum, in order to obtain a beige solid (85 mg, 90%).

[M+H]⁺ experimental=397.22; M theoretical=396.51

NMR-¹H (δ ppm, DMSO): 2.09-2.14 (m, 2H); 2.66-2.75 (m, 2H); 3.2-3.6 (m, 3H); 3.8-4.9 (m, 5H); 5.65-5.80 (m, 1H); 6.48-6.54 (m, 2H); 6.67 (m, 1H); 7.15-7.29 (m, 5H); 7.67 (m, 1H); 8.4-8.9 (m, 3H).

NMR-¹³C (δ ppm, DMSO): 24.65; 31.45; 35.68; 38.07; 47.26; 48.76; 49.25; 51.34; 105.55; 111.49; 125.81; 128.25; 139.38; 141.02; 142.28; 148.45; 166.56.

### EXAMPLE 30

### tert-butyl {(1R)-1-[({(2R)-2-[(tert-butoxycarbonyl)amino]-3-[(4RS)-4-(cyclohexylmethyl)-2-phenyl-6,7-dihydropyrazolo[1,5-a]pyrazin-5(4H)-yl]-3-oxopropyl}diselanyl)methyl]-2-[(4RS)-4-(cyclohexylmethyl)-2-phenyl-6,7-dihydropyrazolo[1,5-a]pyrazin-5(4H)-yl]-2-oxoethyl}carbamate

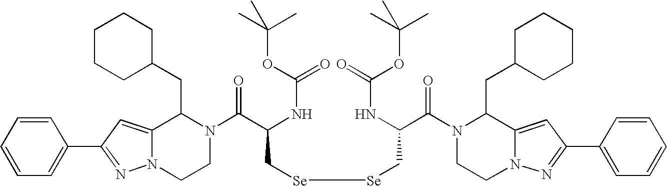

A procedure analogous to the preparation of compound 1f is followed using N,N′-di-Boc-L-selenocystine. A pale solid yellow is obtained.

Melting point: 117-120° C.

[M+H]⁺ experimental monoisotopic=1091.47; M average isotopic=1089.15

NMR-¹H (δ ppm, DMSO): 1.25-1.37 (m, 18H); 0.83-1.74 (m, 26H); 2.9-3.4 (m, 4H); 3.7-4.8 (m, 10H); 5.73 (m, 2H); 6.39-6.55 (m, 2H) 7.25-7.29 (m, 2H) 7.34-7.44 (m, 4H); 7.71-7.76 (m, 4H).

NMR-¹³C (δ ppm, DMSO): 25.40; 25.54; 25.68; 26.06; 31.21; 32.55; 32.75; 32.97; 33.14; 38.89; 47.33; 124.85; 127.42; 128.59; 133.16; 140.82; 149.66; 154.67; 169.48.

### EXAMPLE 31

### {(1R)-1-[({(2R)-2-amino-3-[(4RS)-4-(cyclohexylmethyl)-2-phenyl-6,7-dihydropyrazolo[1,5-a]pyrazin-5(4H)-yl]-3-oxopropyl}diselanyl)methyl]-2-[(4RS)-4-(cyclohexylmethyl)-2-phenyl-6,7-dihydropyrazolo[1,5-a]pyrazin-5(4H)-yl]-2-oxoethyl}amine hydrochloride

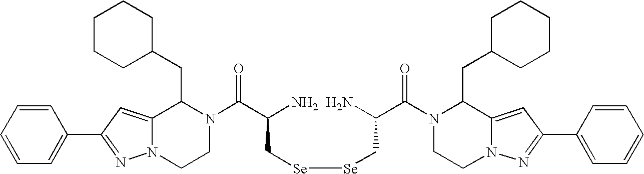

A procedure analogous to the preparation of Example 12 is followed.

Light yellow solid

Melting point: 217-220° C. (dec)

[M+H]⁺ experimental monoisotopic=891.4; M average isotopic=888.92

NMR-¹H (δ ppm, DMSO): 0.88-1.84 (m, 26H); 3.41-3.46 (m, 4H); 3.87-4.78 (m, 10H); 5.60-5.63 (m, 2H); 6.56 (s, 2H); 7.26-7.30 (m, 2H); 7.35-7.39 (m, 4H); 7.75-7.78 (m, 4H); 8.63 (br, 6H).

NMR-¹³C (δ ppm, DMSO): 25.35; 26.07; 29.20; 32.48; 32.88; 32.93; 41.35; 46.96; 49.86; 99.38; 125.04; 127.52; 128.57; 133.08; 139.91; 149.82; 166.07.

### EXAMPLE 32

### (2R)-3-({(2R)-2-amino-3-oxo-3-[(4RS)-4-(2-phenylethyl)-2-pyridin-2-yl-6,7-dihydropyrazolo[1,5-a]pyrazin-5(4H)-yl]propyl}dithio)-1-oxo-1-[(4RS)-4-(2-phenylethyl)-2-pyridin-2-yl-6,7-dihydropyrazolo[1,5-a]pyrazin-5(4H)-yl]propan-2-amine hydrochloride

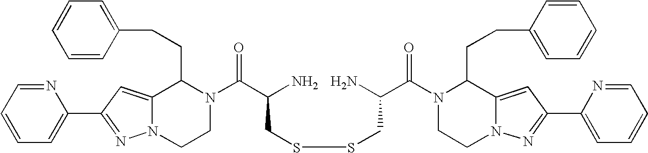

A procedure analogous to the preparation of Example 12 is followed.

White solid—Melting point: 232-234° C. (dec)

[M+2H]²⁺ experimental=407.51; M theoretical=813.07

NMR-¹H (δ ppm, DMSO): 2.12 (m, 4H); 2.66-2.80 (m, 4H); 3.24-5.85 (m, 14H); 5.62-5.85 (m, 2H); 6.67 (s, 2H); 7.16-7.71 (m, 12H); 8.17-8.32 (m, 4H); 8.69-8.91 (m, 10H).

NMR-¹³C (δ ppm, DMSO): 31.02; 31.17; 35.41; 37.70; 47.59; 48.35; 49.04; 102.50; 121.42; 124.17; 125.61; 127.95; 128.07; 140, 35; 140.89; 166, 74.

### Pharmacological Study of the Products of the Invention

### Test Protocol: Characterization of the Anti-Proliferative Activity:

By way of example, the effect of a treatment on three human cell lines DU145, LNCaP and A2058 with the compounds of the examples described previously will be studied. The cell lines DU145 and LNCaP (human prostate cancer cells) and A2058 (human melanoma cancer cells) were acquired from the American Tissue Culture Collection (Rockville, Md., USA). The cells placed in 80 μl of Dulbecco's Modified Eagle medium (Gibco-Brl, Cergy-Pontoise, France) completed with 10% foetal calf serum inactivated by heating (Gibco-Brl, Cergy-Pontoise, France), 50000 units/l of penicillin and 50 mg/l streptomycin (Gibco-Brl, Cergy-Pontoise, France), and 2 mM of glutamin (Gibco-Brl, Cergy-Pontoise, France) were seeded on a 96-well plate on day 0. The cells were treated on day 1 for 96 hours with increasing concentrations of each of the compounds to be tested up to 10 μM. At the end of this period, the quantification of the cell proliferation is evaluated by a colorimetric test based on the cleavage of the tetrazolium salt WST1 by mitochondrial dehydrogenases in the viable cells leading to the formation of formazan (Boehringer Mannheim, Meylan, France). These tests are carried out in duplicate with 8 determinations per tested concentration. For each compound to be tested, the values included in the linear part of the sigmoid were retained for linear regression analysis and used in order to estimate the inhibitory concentration IC₅₀. The products are solubilized in dimethylsulphoxide (DMSO) at 10⁻² M and finally used in culture with 0.1% DMSO.

### Results of the Tests:

a) The compounds of the following examples exhibit an IC₅₀ on the cell proliferation of the DU145 lines of less than or equal to:
20 μM: Examples: 16; 21; 22;
15 Examples: 13; 17; 18; 19; 20; 24
10 μM: Examples: 12

b) The compounds of the following examples exhibit an IC₅₀ on the cell proliferation of the LNCaP lines of less than or equal to:
10 μM: Examples: 3; 14; 15; 16; 17; 20
5 μM: Examples: 18; 19; 21
1 μM: Examples: 12; 13; 22; 24

c) The compounds of the following examples exhibit an IC₅₀ on the cell proliferation of the A2058 lines of less than or equal to:
30 μM: Examples: 14; 16
20 μM: Examples: 15; 17; 18; 19
10 mM: Examples: 12; 13; 20; 21; 22; 24.

1. A compound of formula (I) 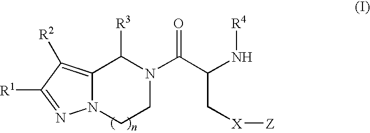
in racemic, enantiomeric or diastereoisomeric form or any combinations thereof, wherein:
Z represents a hydrogen atom or a radical of formula 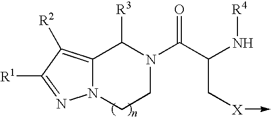
R¹ and R² represent, independently, a hydrogen atom, an aryl or heteroaryl radical, the aryl or heteroaryl radicals being optionally substituted by one or more identical or different substituents chosen from: halo, hydroxy, alkyl, haloalkyl, alkoxy, haloalkoxy, aryl, aryloxy, —NRR′, —C(O)—NRR′, —NR_{N}—C(O)R′, —SO₂—R, —SiRR′R″ or a heterocycloalkyl; or a radical of formula 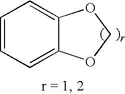
or R¹ and R² form together with the carbon atoms to which they are attached, a cycloalkyl or heterocycloalkyl radical;
R³ represents a (C₁-C₈)alkyl radical or a cycloalkylalkyl, aryl or arylalkyl radical, the aryl group of the aryl and arylalkyl radicals being optionally substituted by one or more identical or different substituents chosen from: halo, hydroxy, alkyl, haloalkyl, alkoxy, haloalkoxy, aryl, aryloxy, —NRR′, —C(O)—NRR′, —NR_{N}—C(O)R′, —SO₂—R, —SiRR′R″ or a heterocycloalkyl;
R_{N} represents a hydrogen atom or an alkyl radical;
R, R′ and R″ represent, independently, an alkyl or aryl radical;
R⁴ represents a hydrogen atom or a radical of formula —CO—O—R⁵;
R⁵ represents an alkyl or arylalkyl radical;
n represents the integer 1 or 2;
X represents a sulfur atom or a selenium atom; or
a pharmaceutically acceptable salt thereof.

2. The compound according to claim 1 wherein Z represents a hydrogen atom; or a pharmaceutically acceptable salt thereof.

3. The compound according to claim 1 wherein Z represents a radical of formula 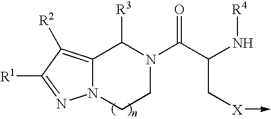
or a pharmaceutically acceptable salt of the latter thereof.

4. The compound according to claim 1, wherein X represents a sulfur atom; or a pharmaceutically acceptable salt thereof.

5. The compound according to claim 1, wherein X represents a selenium atom; or a pharmaceutically acceptable salt thereof.

6. The compound according to claim 1, wherein R² represents a hydrogen atom; or a pharmaceutically acceptable salt thereof.

7. The compound according to claim 1, wherein R⁵ represents an alkyl radical; or a pharmaceutically acceptable salt thereof.

8. The compound according to claim 1, wherein n is equal to 1; or a pharmaceutically acceptable salt thereof.

9. The compound according to claim 1, wherein R¹ represents an aryl or heteroaryl radical, the aryl radical being optionally substituted by one or more identical or different substituents chosen from halo and alkoxy,
or a radical of formula 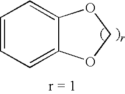
or a pharmaceutically acceptable salt thereof.

10. The compound according to claim 1, wherein R³ represents a C₄-C₈ alkyl, arylalkyl or cycloalkylalkyl radical; or a pharmaceutically acceptable salt thereof.

11. The compound according to claim 1 wherein R² and R⁴ represent a hydrogen atom; or a pharmaceutically acceptable salt thereof.

12. The compound according to claim 11, wherein R³ represents a cycloalkylalkyl or arylalkyl radical; or a pharmaceutically acceptable salt thereof.

13. The compound according to claim 12 wherein R′ represents an aryl or heteroaryl radical, the aryl radical being optionally substituted by one or more identical or different halo substituents; or a pharmaceutically acceptable salt thereof.

14. The compound according to claim 13, wherein R¹ represents a heteroaryl radical; or a pharmaceutically acceptable salt thereof.

15. The compound according to claim 1, wherein:
the cycloalkyl radical of the cycloalkyl and cycloalkylalkyl groups, is a hexyl radical;
the aryl radical of the groups aryl and arylalkyl, is a phenyl radical, and
the heteroaryl is chosen from the following radicals: furyl, thienyl, or pyridinyl; or a pharmaceutically acceptable salt thereof.

16. The compound according to claim 1 wherein said compound is:
tert-butyl {(1R)-1-[({(2R)-2-[(tert-butoxycarbonyl)amino]-3-[(4RS)-4-(cyclohexylmethyl)-2-phenyl-6,7-dihydropyrazolo[1,5-a]pyrazin-5(4H)-yl]-3-oxopropyl}dithio)methyl]-2-[(4RS)-4-(cyclohexylmethyl)-2-phenyl-6,7-dihydropyrazolo[1,5-a]pyrazin-5(4H)-yl]-2-oxoethyl}carbamate;
tert-butyl {(1R)-1-[({(2R)-2-[(tert-butoxycarbonyl)amino]-3-oxo-3-[(4RS)-2-phenyl-4-(2-phenylethyl)-6,7-dihydropyrazolo[1,5-a]pyrazin-5(4H)-yl]propyl}dithio)methyl]-2-oxo-2-[(4RS)-2-phenyl-4-(2-phenylethyl)-6,7-dihydropyrazolo[1,5-a]pyrazin-5(4H)-yl]ethyl}carbamate;
tert-butyl {(1R)-1-[({(2R)-2-[tert-butoxycarbonyl)amino]-3-oxo-3-[(4RS)-4-(2-phenylethyl)-2-pyridin-4-yl-6,7-dihydropyrazolo[1,5-a]pyrazin-5(4H)-yl]propyl}dithio)methyl]-2-oxo-2-[(4RS)-4-(2-phenylethyl)-2-pyridin-4-yl-6,7-dihydropyrazolo[1,5-a]pyrazin-5(4H)-yl]ethyl}carbamate;
tert-butyl {(1R)-1-[({(2R)-2-[(tert-butoxycarbonyl)amino]-3-oxo-3-[(4RS)-4-(2-phenylethyl)-2-pyridin-3-yl-6,7-dihydropyrazolo[1,5-a]pyrazin-5(4H)-yl]propyl}dithio)methyl]-2-oxo-2-[(4RS)-4-(2-phenylethyl)-2-pyridin-3-yl-6,7-dihydro pyrazolo[1,5-a]pyrazin-5(4H)-yl]ethyl}carbamate;
tert-butyl {(1R)-2-[(4RS)-2-(1,3-benzodioxol-5-yl)-4-(2-phenylethyl)-6,7-dihydropyrazolo[1,5-a]pyrazin-5(4H)-yl]-1-[({(2R)-3-[(4RS)-2-(1,3-benzodioxol-5-yl)-4-(2-phenylethyl)-6,7-dihydropyrazolo[1,5-a]pyrazin-5(4H)-yl]-2-[(tert-butoxycarbonyl)amino]-3-oxopropyl}dithio)methyl]-2-oxoethyl}carbamate;
tert-butyl {(1R)-1-[({(2R)-2-[(tert-butoxycarbonyl)amino]-3-oxo-3-[(4RS)-4-(2-phenylethyl)-2-(3,4,5-trimethoxyphenyl)-6,7-dihydropyrazolo[1,5-a]pyrazin-5(4H)-yl]propyl}dithio)methyl]-2-oxo-2-[(4RS)-4-(2-phenylethyl)-2-(3,4,5-trimethoxyphenyl)-6,7-dihydropyrazolo[1,5-a]pyrazin-5(4H)-yl]ethyl}carbamate;
tert-butyl {(1R)-1-[({(2R)-2-[(tert-butoxycarbonyl)amino]-3-oxo-3-[(4RS)-4-(2-phenylethyl)-2-(2-thienyl)-6,7-dihydropyrazolo[1,5-a]pyrazin-5(4H)-yl]propyl}dithio)methyl]-2-oxo-2-[(4RS)-4-(2-phenylethyl)-2-(2-thienyl)-6,7-dihydropyrazolo[1,5-a]pyrazin-5(4H)-yl]ethyl}carbamate;
tert-butyl {(1R)-1-[({(2R)-2-[(tert-butoxycarbonyl)amino]-3-[(4RS)-2-(2-furyl)-4-(2-phenylethyl)-6,7-dihydropyrazolo[1,5-a]pyrazin-5(4H)-yl]-3-oxopropyl}dithio)methyl]-2-[(4RS)-2-(2-furyl)-4-(2-phenylethyl)-6,7-dihydropyrazolo[1,5-a]pyrazin-5(4H)-yl]-2-oxoethyl}carbamate;
tert-butyl {(1R)-1-[({(2R)-2-[(tert-butoxycarbonyl)amino]-3-oxo-3-[(4RS)-4-pentyl-2-phenyl-6,7-dihydropyrazolo[1,5-a]pyrazin-5(4H)-yl]propyl}dithio)methyl]-2-oxo-2-[(4RS)-4-pentyl-2-phenyl-6,7-dihydropyrazolo[1,5-a]pyrazin-5(4H)-yl]ethyl}carbamate;
tert-butyl {(1R)-1-[({(2R)-2-[(tert-butoxycarbonyl)amino]-3-[(4RS)-4-butyl-2-phenyl-6,7-dihydropyrazolo[1,5-a]pyrazin-5(4H)-yl]-3-oxopropyl}dithio)methyl]-2-[(4RS)-4-butyl-2-phenyl-6,7-dihydropyrazolo[1,5-a]pyrazin-5(4H)-yl]-2-oxoethyl}carbamate;
tert-butyl {(1R)-1-[({(2R)-2-[(tert-butoxycarbonyl)amino]-3-[(4RS)-2-(2,4-dichlorophenyl)-4-(2-phenylethyl)-6,7-dihydropyrazolo[1,5-a]pyrazin-5(4H)-yl]-3-oxopropyl}dithio)methyl]-2-[(4RS)-2-(2,4-dichlorophenyl)-4-(2-phenylethyl)-6,7-dihydropyrazolo[1,5-a]pyrazin-5(4H)-yl]-2-oxoethyl}carbamate;
(2R)-3-({(2R)-2-amino-3-[(4RS)-4-(cyclohexylmethyl)-2-phenyl-6,7-dihydropyrazolo[1,5-a]pyrazin-5(4H)-yl]-3-oxopropyl}dithio)-1-[(4RS)-4-(cyclohexylmethyl)-2-pheny;1-6,7-dihydropyrazolo[1,5-a]pyrazin-5(4H)-yl]-1-oxopropan-2-amine hydrochloride;
(2R)-3-({(2R)-2-amino-3-oxo-3-[(4RS)-2-phenyl-4-(2-phenylethyl)-6,7-dihydro pyrazolo[1,5-a]pyrazin-5(4H)-yl]propyl}dithio)-1-oxo-1-[(4RS)-2-phenyl-4-(2-phenylethyl)-6,7-dihydropyrazolo[1,5-a]pyrazin-5(4H)-yl]propan-2-amine hydrochloride;
(2R)-3-({(2R)-2-amino-3-oxo-3-[(4RS)-4-(2-phenylethyl)-2-pyridin-4-yl-6,7-dihydropyrazolo[1,5-a]pyrazin-5(4H)-yl]propyl}dithio)-1-oxo-1-[(4RS)-4-(2-phenylethyl)-2-pyridin-4-yl-6,7-dihydropyrazolo[1,5-a]pyrazin-5(4H)-yl]propan-2-amine hydrochloride;
(2R)-3-({(2R)-2-amino-3-oxo-3-[(4RS)-4-(2-phenylethyl)-2-pyridin-3-yl-6,7-dihydropyrazolo[1,5-a]pyrazin-5(4H)-yl]propyl}dithio)-1-oxo-1-[(4RS)-4-(2-phenylethyl)-2-pyri din-3-yl-6,7-dihydropyrazolo[1,5-a]pyrazin-5(4H)-yl]propan-2-amine hydrochloride;
(2R)-3-({(2R)-2-amino-3-[(4RS)-2-(1,3-benzodioxol-5-yl)-4-(2-phenylethyl)-6,7-dihydropyrazolo[1,5-a]pyrazin-5(4H)-yl]-3-oxopropyl}dithio)-1-[(4RS)-2-(1,3-benzodioxol-5-yl)-4-(2-phenylethyl)-6,7-dihydropyrazolo[1,5-a]pyrazin-5(4H)-yl]-1-oxopropan-2-amine hydrochloride;
(2R)-3-({(2R)-2-amino-3-oxo-3-[(4RS)-4-(2-phenylethyl)-2-(3,4,5-trimethoxyphenyl)-6,7-dihydropyrazolo[1,5-a]pyrazin-5(4H)-yl]propyl}dithio)-1-oxo-1-[(4RS)-4-(2-phenylethyl)-2-(3,4,5-trimethoxyphenyl)-6,7-dihydropyrazolo[1,5-a]pyrazin-5(4H)-yl]propan-2-amine hydrochloride;
(2R)-3-({(2R)-2-amino-3-oxo-3-[(4RS)-4-(2-phenylethyl)-2-(2-thienyl)-6,7-dihydropyrazolo[1,5-a]pyrazin-5(4H)-yl]propyl}dithio)-1-oxo-1-[(4RS)-4-(2-phenylethyl)-2-(2-thienyl)-6,7-dihydropyrazolo[1,5-a]pyrazin-5(4H)-yl]propan-2-amine hydrochloride;
(2R)-3-({(2R)-2-amino-3-[(4RS)-2-(2-furyl)-4-(2-phenylethyl)-6,7-dihydropyrazolo[1,5-a]pyrazin-5(4H)-yl]-3-oxopropyl}dithio)-1-[(4RS)-2-(2-furyl)-4-(2-phenylethyl)-6,7-dihydropyrazolo[1,5-a]pyrazin-5(4H)-yl]-1-oxopropan-2-amine hydrochloride;
(2R)-3-({(2R)-2-amino-3-oxo-3-[(4RS)-4-pentyl-2-phenyl-6,7-dihydropyrazolo[1,5-a]pyrazin-5(4H)-yl]propyl}dithio)-1-oxo-1-[(4RS)-4-pentyl-2-phenyl-6,7-dihydropyrazolo[1,5-a]pyrazin-5(4H)-yl]propan-2-amine hydrochloride;
(2R)-3-({(2R)-2-amino-3-[(4RS)-4-butyl-2-phenyl-6,7-dihydropyrazolo[1,5-a]pyrazin-5(4H)-yl]-3-oxopropyl}dithio)-1-[(4RS)-4-butyl-2-phenyl-6,7-dihydropyrazolo[1,5-a]pyrazin-5(4H)-yl]-1-oxopropan-2-amine hydrochloride;
(2R)-3-({(2R)-2-amino-3-[(4RS)-2-(2,4-dichlorophenyl)-4-(2-phenylethyl)-6,7-dihydropyrazolo[1,5-a]pyrazin-5(4H)-yl]-3-oxopropyl}dithio)-1-[(4RS)-2-(2,4-dichlorophenyl)-4-(2-phenylethyl)-6,7-dihydropyrazolo[1,5-a]pyrazin-5(4H)-yl]-1-oxopropan-2-amine hydrochloride;
tert-butyl {(1R)-1-[({(2R)-2-[(tert-butoxycarbonyl)amino]-3-[(4S)-4-(cyclohexylmethyl)-2-phenyl-6,7-dihydropyrazolo[1,5-a]pyrazin-5(4H)-yl]-3-oxopropyl}dithio)methyl]-2-[(4S)-4-(cyclohexylmethyl)-2-phenyl-6,7-dihydropyrazolo[1,5-a]pyrazin-5(4H)-yl]-2-oxoethyl)carbamate;
(2R)-3-({(2R)-2-amino-3-[(4S)-4-(cyclohexylmethyl)-2-phenyl-6,7-dihydropyrazolo[1,5-a]pyrazin-5(4H)-yl]-3-oxopropyl}dithio)-1-[(4S)-4-(cyclohexylmethyl)-2-phenyl-6,7-dihydropyrazolo[1,5-a]pyrazin-5(4H)-yl]-1-oxopropan-2-amine hydrochloride;
tert-butyl {(1R)-1-[({(2R)-2-[(tert-butoxycarbonyl)amino]-3-[(4R)-4-(cyclohexylmethyl)-2-phenyl-6,7-dihydropyrazolo[1,5-a]pyrazin-5(4H)-yl]-3-oxopropyl}dithio)methyl]-2-[(4R)-4-(cyclohexylmethyl)-2-phenyl-6,7-dihydropyrazolo[1,5-a]pyrazin-5(4H)-yl]-2-oxoethyl}carbamate;
(1R)-3-({(2R)-2-amino-3-[(4R)-4-(cyclohexylmethyl)-2-phenyl-6,7-dihydropyrazolo[1,5-a]pyrazin-5(4H)-yl]-3-oxopropyl}dithio)-1-[(4R)-4-(cyclohexylmethyl)-2-phenyl-6,7-dihydropyrazolo[1,5-a]pyrazin-5(4H)-yl]-1-oxopropan-2-amine hydrochloride;
tert-butyl {(1R)-1-[({(2R)-2-[(tert-butoxycarbonyl)amino]-3-[(4RS)-4-(cyclohexylmethyl)-2-phenyl-7,8-dihydro-4H-pyrazolo[1,5-a][1,4]diazepin-5(6H)-yl]-3-oxopropyl}dithio)methyl]-2-[(4RS)-4-(cyclohexylmethyl)-2-phenyl-7,8-dihydro-4H-pyrazolo[1,5-a][1,4]diazepin-5(6H)-yl]-2-oxoethyl}carbamate;
{(1R)-1-[({(2R)-2-amino-3-[(4RS)-4-(cyclohexylmethyl)-2-phenyl-7,8-dihydro-4H-pyrazolo[1,5-a][1,4]diazepin-5(6H)-yl]-3-oxopropyl}dithio)methyl]-2-[(4RS)-4-(cyclohexylmethyl)-2-phenyl-7,8-dihydro-4H-pyrazolo[1,5-a][1,4]diazepin-5(6H)-yl]-2-oxoethyl}amine hydrochloride;
(2R)-2-amino-3-[(4RS)-2-(2-furyl)-4-(2-phenylethyl)-6,7-dihydropyrazolo[1,5-a]pyrazin-5(4H)-yl]-3-oxopropane-1-thiol hydrochloride;
tert-butyl {(1R)-1-[({(2R)-2-[(tert-butoxycarbonyl)amino]-3-[(4RS)-4-(cyclohexylmethyl)-2-phenyl-6,7-dihydropyrazolo[1,5-a]pyrazin-5(4H)-yl]-3-oxopropyl}diselanyl)methyl]-2-[(4RS)-4-(cyclohexylmethyl)-2-phenyl-6,7-dihydropyrazolo[1,5-a]pyrazin-5(4H)-yl]-2-oxoethyl}carbamate;
{(1R)-1-[({(2R)-2-amino-3-[(4RS)-4-(cyclohexylmethyl)-2-phenyl-6,7-dihydro pyrazolo[1,5-a]pyrazin-5(4H)-yl]-3-oxopropyl}diselanyl)methyl]-2-[(4RS)-4-(cyclohex ylmethyl)-2-phenyl-6,7-dihydropyrazolo[1,5-a]pyrazin-5(4H)-yl]-2-oxoethyl}amine hydrochloride; or
(2R)-3-({(2R)-2-amino-3-oxo-3-[(4RS)-4-(2-phenylethyl)-2-pyridin-2-yl-6,7-dihydropyrazolo[1,5-a]pyrazin-5(4H)-yl]propyl}dithio)-1-oxo-1-[(4RS)-4-(2-phenylethyl)-2-pyridin-2-yl-6,7-dihydropyrazolo[1,5-a]pyrazin-5(4H)-yl]propan-2-amine hydrochloride; or a pharmaceutically acceptable salt thereof.

17. A process for the preparation of a compound of formula (I) 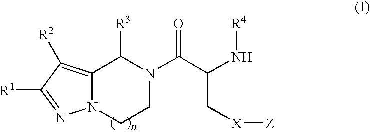
in racemic, enantiomeric or diastereoisomeric form or any combinations thereof, wherein:
Z represents a hydrogen atom or a radical of formula 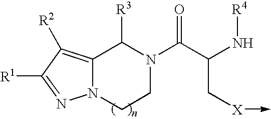
R¹ and R² represent, independently, a hydrogen atom, an aryl or heteroaryl radical, the aryl or heteroaryl radicals being optionally substituted by one or more identical or different substituents chosen from: halo, hydroxy, alkyl, haloalkyl, alkoxy, haloalkoxy, aryl, aryloxy, —NRR′, —C(O)—NRR′, —NR_{N}—C(O)R′, —SO₂—R, —SiRR′R″ or a heterocycloalkyl; or a radical of formula 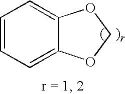
or R¹ and R² form together with the carbon atoms to which they are attached, a cycloalkyl or heterocycloalkyl radical;
R³ represents a (C₁-C₈)alkyl radical or a cycloalkylalkyl, aryl or arylalkyl radical, the aryl group of the aryl and arylalkyl radicals being optionally substituted by one or more identical or different substituents chosen from: halo, hydroxy, alkyl, haloalkyl, alkoxy, haloalkoxy, aryl, aryloxy, —NRR′, —C(O)—NRR′, —NR_{N}—C(O)R′, —SO₂—R, —SiRR′R″ or a heterocycloalkyl;
R_{N} represents a hydrogen atom or an alkyl radical;
R, R′ and R″ represent, independently, an alkyl or aryl radical;
R⁴ represents a hydrogen atom or a radical of formula —CO—O—R⁵;
R⁵ represents an alkyl or arylalkyl radical;
n represents the integer 1 or 2;
X represents a sulfur atom or a selenium atom; or a pharmaceutically acceptable salt thereof, wherein:
a compound of formula (VI) 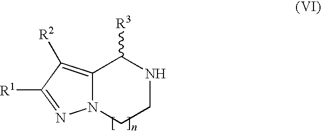
is reacted with a carboxylic acid of formula (VII) 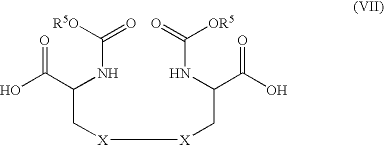
under peptide coupling conditions, at a temperature of between 0° C. and 30° C., in an aprotic solvent, in order to obtain:
the compound of formula (I) in which Z is different from the hydrogen atom and R⁴ represents the —CO—O—R⁵ radical,
compound of formula (I) in which R⁴ represents the —CO—O—R⁵ radical which can be deprotected, in order to obtain the compound of formula (I) in which Z is different from the hydrogen atom and R⁴ represents the hydrogen atom, or
the compound of formula (I) in which Z is different from the hydrogen atom which can be reduced in order to obtain the corresponding compound of formula (I) in which Z represents the hydrogen atom.

18. A pharmaceutical composition comprising a compound of formula (I) according to claim 1 or a pharmaceutically acceptable salt thereof, and at least one pharmaceutically acceptable excipient.

19. A drug compound of formula (I) according to claim 1 or a pharmaceutically acceptable salt thereof.

20. A method of treating or preventing a disease or a disorder comprising the administration of at least one of the compounds of formula (I) according to claim 1, or a pharmaceutically acceptable salt thereof, wherein the disease or disorder is cancer, non-tumorous proliferative disease, neurodegenerative disease, parasitic disease, viral infection, spontaneous alopecia, alopecia induced by exogenous products, radiation-induced alopecia, auto-immune disease, graft rejection, inflammatory disease, an allergic disease, or pain.

21. The method according to claim 20, wherein the disease or disorder treated or prevented is cancer.

22. The method according to claim 21, wherein the cancer treated or prevented is colon, rectum, stomach, lungs, pancreas, kidney, testicles, breast, uterus, ovary, prostate, skin, bone, spinal cord, neck, tongue, or head.

23. A compound of formula (VI) 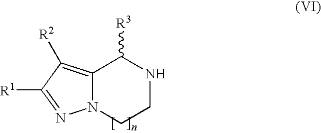
in racemic or enantiomeric form or any combinations thereof, wherein:
R¹ and R² represent, independently, a hydrogen atom, an aryl or heteroaryl radical, the aryl or heteroaryl radicals being optionally substituted by one or more identical or different substituents chosen from: halo, hydroxy, alkyl, haloalkyl, alkoxy, haloalkoxy, aryl, aryloxy, —NRR′, —C(O)—NRR′, —NR_{N}—C(O)R″, —SO₂—R, —SiRR′R″ or a heterocycloalkyl; or a radical of formula 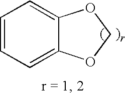
or R¹ and R² form together with the carbon atoms to which they are attached, a cycloalkyl or heterocycloalkyl radical;
R³ represents a (C₁-C₈)alkyl radical or a cycloalkylalkyl, aryl or arylalkyl radical, the aryl group of the aryl and arylalkyl radicals being optionally substituted by one or more identical or different substituents chosen from: halo, hydroxy, alkyl, haloalkyl, alkoxy, haloalkoxy, aryl, aryloxy, —NRR′, —C(O)—NRR′, —NR_{N}—C(O)R′, —SO₂—R, —SiRR′R″ or a heterocycloalkyl;
R_{N} represents a hydrogen atom or an alkyl radical;
R, R′ and R″ represent, independently, an alkyl or aryl radical;
R⁴ represents a hydrogen atom or a radical of formula —CO—O—R⁵;
R⁵ represents an alkyl or arylalkyl radical;
n represents the integer 1 or 2;
X represents a sulfur atom or a selenium atom; or a pharmaceutically acceptable salt thereof.

24. A process for the preparation of a compound of formula (VI) according to claim 23, comprising the steps of:
(a) subjecting to acidic conditions the compound of formula (IV) 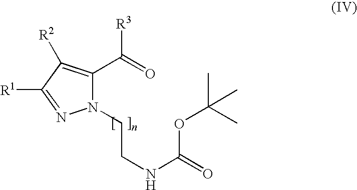
to release the amine function,
(b) neutralizing the compound created in step (a), to form a compound of formula (V) 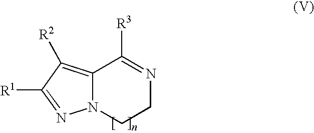
to form an imine function of the compound of formula (V), and
(c) reducing the compound formed in step (b) to produce the cyclic amine of formula (VI).

25. The method of claim 21 wherein the cancer treated or prevented is a sarcoma, carcinoma, fibroadenoma, neuroblastoma, leukemia, or melanomas.
